(19) United States
(12) Patent Application Publication
(10) Pub. No.: US 2024/0307528 A1
(43) Pub. Date: Sep. 19, 2024

(54) METHODS OF BOOSTING AN IMMUNE RESPONSE AGAINST HIV USING COMMENSAL MICROBE ANTIGENS

(71) Applicant: VANDERBILT UNIVERSITY, Nashville, TN (US)

(72) Inventors: Lauren M. Walker, Nashville, TN (US); Ivelin Stefanov Georgiev, Nashville, TN (US)

(21) Appl. No.: 18/606,343

(22) Filed: Mar. 15, 2024

Related U.S. Application Data
(60) Provisional application No. 63/490,661, filed on Mar. 16, 2023.

(51) Int. Cl. A61K 39/295; A61K 39/00; A61P 31/18; C12N 7/00

(52) U.S. Cl. CPC A61K 39/295; A61P 31/18; C12N 7/00; A61K 2039/55566; A61K 2039/575; C12N 2740/16034

(57) ABSTRACT

The present disclosure relates to HIV vaccines and methods of use thereof. Disclosed herein are methods of preventing an infection from a human immunodeficiency virus (HIV) using a commensal microbe antigen. Further disclosed herein is a method of boosting an immune response against a human immunodeficiency virus (HIV), the method comprising administering to a subject an effective amount of a first composition comprising a commensal microbe antigen and an effective amount of a second composition comprising an HIV antigen.

### CROSS-REFERENCE TO RELATED APPLICATIONS

This application claims priority to, and the benefit of, U.S. Provisional Patent Application No. 63/490,661, filed Mar. 16, 2023, which is incorporated by reference herein in its entirety.

### STATEMENT REGARDING FEDERALLY SPONSORED RESEARCH

This invention was made with Government Support under Grant Nos. R01AI131722 and R01AI152693 awarded by the National Institutes of Health. The Government has certain rights in the invention.

### REFERENCE TO SEQUENCE LISTING

The sequence listing submitted on Mar. 15, 2024, as an .XML file entitled “10644-160US1_ST26.xml” created on Mar. 15, 2024, and having a file size of 110,834 bytes is hereby incorporated by reference pursuant to 37 C.F.R. § 1.52(e)(5).

### FIELD

The present disclosure relates to HIV vaccines and methods of use thereof.

### BACKGROUND

Over four decades since the start of the AIDS epidemic, HIV-1 infection remains a major public health threat with millions of new infections and thousands of deaths occurring globally each year. Despite considerable progress in drugs used to treat infection, such as antiretrovirals, no licensed vaccine candidate exists to prevent HIV-1 infection. A key to stopping the AIDS epidemic is to prevent new HIV-1 infections from occurring. Therefore, there is an urgent need for a preventative HIV-1 vaccine, however, no vaccine candidate has proven successful at preventing infection. A critical barrier to the development of an effective vaccine has been the heterogeneity of immune responses elicited by clinical trial participants. Therefore, defining host specific factors that influence vaccine response proves critical in the development of an effective HIV-1 vaccine.

### SUMMARY

The present disclosure also provides methods of developing, generating, and/or improving vaccines.

In one aspect, disclosed herein is a method of preventing an infection from a human immunodeficiency virus (HIV), the method comprising administering to a subject an effective amount of a first composition comprising a commensal microbe antigen and an effective amount of a second composition comprising an HIV antigen.

In one aspect, disclosed herein is a method of boosting an immune response against a human immunodeficiency virus (HIV), the method comprising administering to a subject an effective amount of a first composition comprising a commensal microbe antigen and an effective amount of a second composition comprising an HIV antigen.

In some embodiments, the first composition is administered before the second composition. In some embodiments, the first composition is simultaneously administered with the second composition.

In some embodiments, the commensal microbe antigen is derived from an intestinal microbe. In some embodiments, the intestinal microbe comprises Limosilactobacilius reuteri (L. reuteri) or Bacteroides thetaiotaomicron (B. thetaiotaomicron).

In some embodiments, the commensal microbe antigen comprises at least 80% sequence identity to SEQ ID NO: 7. In some embodiments, the commensal microbe antigen comprises at least 90% sequence identity to SEQ ID NO: 7. In some embodiments, the commensal microbe antigen comprises SEQ ID NO: 7. In some embodiments, the commensal microbe antigen comprises Elongation Factor Tu (EF-Tu). In some embodiments, EF-Tu comprises SEQ ID NO: 59, or a fragment thereof.

In some embodiments, the HIV antigen is derived from an HIV-1 virus. In some embodiments, the HIV antigen comprises an HIV glycoprotein, or a fragment thereof. In some embodiments, the HIV antigen comprises SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, or a fragment thereof. In some embodiments, the HIV glycoprotein comprises a whole HIV envelope (Env) protein. In some embodiments, a fragment of the HIV glycoprotein comprises a CD4 binding (CD4bs), a CD4 induced site (CD4i), a VIV2 loop region (V1V2), a variable region 3 (V3), a membrane proximal external region (MPER), or a fusion peptide (FP).

In some embodiments, the method further comprises administering an adjuvant. In some embodiments, the adjuvant comprises a water-in-oil adjuvant.

In some embodiments, the commensal microbe antigen and the HIV antigen enhances an immune response against the HIV relative to an HIV antigen alone. In some embodiments, the immune response comprises an increased HIV antibody response relative to a control. In some embodiments, the method prevents infection from the HIV.

### BRIEF DESCRIPTION OF FIGURES

The accompanying figures, which are incorporated in and constitute a part of this specification, illustrate several aspects described below.

FIG. 1 shows that the HIV-1 specific monoclonal antibodies (mAbs) bind to whole cell lysate from the commensal microbe Limosilactobacillus reuteri. Native western blot analysis of 25 μg of L. reuteri lysate probed against 10 μg/ml of antibody. Native protein ladder is shown on the left. Antibodies and epitope information are under each blot.

FIG. 2 shows that HIV-1 specific (mAbs) are probed for binding to Bacteroides thetaiotaomicron and Akkermansia muciniphila. Native western blot analysis. 25 μg of lysate probed against 10 μg/ml of antibody. Native protein ladder is shown on the left. Antibodies and epitope information are under each blot. Top is B. thetaiotaomicron and bottom is A. muciniphila.

FIGS. 3A and 3B show HIV-1 antibodies show diverse levels of polyreactivity. FIG. 3A shows the antibody binding to a panel of autoantigens using AtheNA multiplex assay. Y-axis is arbitrary units (AU), Positive=>120 at 25 μg/ml. Antibody concentration on the X-axis. FIG. 3B shows the antibody binding to cardiolipin. QUANTA Lite ACA IgG III. Y-axis IgG phospholipid units. Positive=>=20 at 50 μg/ml. Antibody concentration on the X-axis

FIGS. 4A and 4B show the HIV-1 antibody panel shows low cross-reactivity to bacterial, parasitic and viral antigens. FIG. 4A shows the ELISA binding curves to viral, bacteria and parasitic antigens are shown for HIV-1 mAbs. Y-axis absorbance at 450 nm. mAb concentration is on the X-axis in ug/mL. FIG. 4B shows the Heat map of ELISA AUC values for ELISA binding curves in FIG. 4A.

FIGS. 5A and 5B show the immunoprecipitation and computational analysis to identify binding partners of HIV-1 monoclonal antibody CH58. FIG. 5A shows the schematic of immunoprecipitation using Dynabeads M-270 Epoxy beads. FIG. 5B shows the schematic of computational analysis of mass spectrometry hits. The CH58 epitope region of 6352 strains of HIV-1 was aligned to proteins indentified through mass spectrometry.

FIGS. 6A, 6B, and 6C show the Elongation Factor Tu identified among possible targets as the binding partner of CH58. FIG. 6A shows the EF-Tu pepides matched to CH58 Env epitope region (left). ELISA binding curves for peptides shown for CH58. Peptide 7 is a positve control. FIG. 6B shows the species representative genomes from UHGG collection downloaded and aligned to the amino acid sequence of EF-Tu (L. reuteri. Strain JCM 1112). The tree was visualized and annotated using Interactive Tree of Life with phylum for reference. Percent identity to reference in blue. FIG. 6C shows the violin plot for percent identity to reference (Y-axis). Each dot represents one species representative from that phyla. ELISA curves and violin plots generated in GraphPad Prism.

FIG. 7 shows the study design for animal immunizations. FIG. 7 also shows the immunization schedule and antigen groups.

FIGS. 8A and 8B show that EF-Tu prime immunization alters development of HIV-1 antigen specific antibody response. FIG. 8A shows the ELISA binding curves to HIV-1 Env and EF-Tu proteins are shown for both immunization groups at time 0 (pre-bleed) and time 4 (terminal bleed). Y-axis absorbance at 450 nm. mAb concentration is on the X-axis in μg/ml. FIG. 8B shows the EC₅₀ (concentration of serum at which half-maximal repsonse is observed) against each antigen in both groups at both time points. Statistical significance was determined by Mann-Whitney U test. Data analyzed in GraphPad Prism.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, 100% or more increase so long as the increase is statistically significant.

As used herein, “enhance”, “enhanced”, “enhancement”, “enhancing”, and any grammatical variations thereof as used herein, refers to an act of intensifying, increasing, or further improving the quality, value, or extent of a biological function, composition, compound, cell, or tissue. Herein, the terms “boost”, “optimize”, and any grammatical variations thereof, can be used interchangeably with the term “enhance” and any grammatical variations thereof.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

A “control” is an alternative subject or sample used in an experiment for comparison purposes. A control can be “positive” or “negative.”

The term “amino acid,” includes but is not limited to amino acids contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

Reference also is made herein to peptides, polypeptides, proteins, and compositions comprising peptides, polypeptides, and proteins. As used herein, a polypeptide and/or protein is defined as a polymer of amino acids, typically of length≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110).

The peptides, polypeptides, and proteins disclosed herein may be modified to include non-amino acid moicties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine, or histidine).

The phrases “percent identity” and “ % identity,” as applied to polypeptide sequences, refer to the percentage of residue matches between at least two polypeptide sequences aligned using a standardized algorithm. Methods of polypeptide sequence alignment are well-known. Some alignment methods consider conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length may be used to describe a length over which percentage identity may be measured.

The term “variant” means a polypeptide derived from a parent polypeptide by one or more (several) alteration(s), i.e., a substitution, insertion, and/or deletion, at one or more (several) positions. A substitution means a replacement of an amino acid occupying a position with a different amino acid; a deletion means removal of an amino acid occupying a position; and an insertion means adding 1 or more, such as 1, 2, 3, 4, 5, 6, 7, 8, 9 or 10, preferably 1-3 amino acids immediately adjacent an amino acid occupying a position. In relation to substitutions, ‘immediately adjacent’ may be to the N-side (‘upstream’) or C-side (‘downstream’) of the amino acid occupying a position (‘the named amino acid’). Therefore, for an amino acid named/numbered ‘X,’ the insertion may be at position ‘X+1’ (‘downstream’) or at position ‘X−1’ (‘upstream’).

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polypeptide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polypeptide.

A variant polypeptide may have substantially the same functional activity as a reference polypeptide. For example, a variant polypeptide may exhibit or more biological activities associated with binding a ligand and/or binding DNA at a specific binding site.

Variants comprising a fragment of a reference amino acid sequence are contemplated herein. A “fragment” is a portion of an amino acid sequence which is identical in sequence to but shorter in length than the reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous amino acid residues of a reference polypeptide, respectively. Fragments may be preferentially selected from certain regions of a molecule, for example the N-terminal region and/or the C-terminal region of a polypeptide. The term “at least a fragment” encompasses the full length polypeptide.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “antibody” is used in the broadest sense, and specifically covers monoclonal antibodies (including full length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end.

The term “antibody fragment” refers to a portion of a full-length antibody, generally the target binding or variable region. Examples of antibody fragments include Fab, Fab′, F(ab′)₂ and Fv fragments. The phrase “functional fragment or analog” of an antibody is a compound having qualitative biological activity in common with a full-length antibody. For example, a functional fragment or analog of an anti-IgE antibody is one which can bind to an IgE immunoglobulin in such a manner so as to prevent or substantially reduce the ability of such molecule from having the ability to bind to the high affinity receptor, FcεRI. As used herein, “functional fragment” with respect to antibodies, refers to Fv, F(ab) and F(ab′)₂ fragments. An “Fv” fragment is the minimum antibody fragment which contains a complete target recognition and binding site. This region consists of a dimer of one heavy and one light chain variable domain in a tight, non-covalent association (V_{H}-V_{L} dimer). It is in this configuration that the three CDRs of each variable domain interact to define a target binding site on the surface of the V_{H}-V_{L} dimer. Collectively, the six CDRs confer target binding specificity to the antibody. However, even a single variable domain (or half of an Fv comprising only three CDRs specific for a target) has the ability to recognize and bind target, although at a lower affinity than the entire binding site. “Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the sFv to form the desired structure for target binding.

The term “monoclonal antibody” as used herein refers to an antibody obtained from a substantially homogencous population of antibodies, i.e., the individual antibodies within the population are identical except for possible naturally occurring mutations that may be present in a small subset of the antibody molecules.

A “vaccine” refers to a biological preparation that provides active acquired immunity to a particular infectious diseases caused by a virus, bacteria, parasite, or any other microorganisms. Vaccines typically comprise an agent or several agents, also referred to as antigens, resembling the disease-causing microorganism and is often made from weakened or killed forms of the microbe, its toxins, or its surface proteins/peptides. Vaccines are also made to comprise additional components, such as adjuvants, preservatives, and/or stabilizers to boost the immune response, improve safety, and improve vaccine storage.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEENTM (ICI, Inc .; Bridgewater, New Jersey), polyethylene glycol (PEG), and PLURONICS (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

“Effective amount” encompasses, without limitation, an amount that can ameliorate, reverse, mitigate, prevent, or diagnose a symptom or sign of a medical condition or disorder (e.g., HIV-1 infection). Unless dictated otherwise, explicitly or by context, an “effective amount” is not limited to a minimal amount sufficient to ameliorate a condition. The severity of a disease or disorder, as well as the ability of a treatment to prevent, treat, or mitigate, the disease or disorder can be measured, without implying any limitation, by a biomarker or by a clinical parameter.

As used herein, the term infection refers to the invasion of tissues by pathogens, their multiplication, and reaction of host tissues to the infectious agent and any toxins they release. Infections can be caused by a wide range of pathogen, most common are bacteria and viruses.

A “commensal microbe” refers to a type of microbe, including but not limited to intestinal bacteria, that reside on either the surface of the body or within mucosal tissues without causing harm to the host, such as for example a human host. It should be noted that the commensal microbes living in harmony with most hosts mostly comprise of populations of bacteria.

A “virus” is a microscopic infectious agent that replicates only inside the living cells of an organism. Viruses can infect all life forms, including mammalian and non-mammalian animals, plants, and other microorganisms. A complete virus, also known as a virion, consists of nucleic acid genetic material surrounded by a protective coat of protein called a capsid. Virus can have a lipid envelope derived from the infected host cell membrane. In general, there are five morphological virus types including helical, icosahedral, prolate, enveloped, and complex virus. A virus can either have a DNA or RNA genome, though a vast majority have RNA genomes. Irrespective of the type of nucleic acid genome, a viral genome can be either a single-stranded genome or a double-stranded genome.

An “epitope” or “antigenic determinant” refer to the part of an antigen, a molecular structure, or foreign particulate that can bind to a specific antibody or T-cell receptor. The presence of antigens or epitopes of antigens within a host can illicit an immune response.

An “antigen” refers to a molecule, moiety, foreign particulate matter, or an allergen that can bind to a specific antibody or T cell receptor. The presence of antigens within a host can illicit an immune response against said molecule, moiety, foreign particulate matter, or allergen.

An “adjuvant” refers to a drug, molecule, substance, or a combination thereof that is used to increase the efficacy or potency of certain therapeutic agents, such as for example vaccines and/or antibodies. “Adjuvant(s)” are often at least one ingredient used in some vaccines that help create a stronger immune response in the host receiving said vaccine.

### Methods

At present, there are 38.4 million people worldwide infected with Human Immunodeficiency virus (HIV-1), and it is estimated that between 0.5 million-0.9 million people die of HIV-1-related illness each year. HIV-1 is a retrovirus meaning it is able to integrate its DNA into its host's genome, thereby causing a lifelong infection. Despite considerable progress in antiretroviral therapy (ART) and prevention strategies such as pre-exposure prophylaxis (PrEP), there were still 1.5 million new HIV-1 infections in 2021. Studies have proven the effectiveness of ART on lowering HIV-1 transmission rates, however 25% of infected individuals are not accessing treatment. A key to stopping the HIV-1 epidemic is to prevent new infections from occurring. Therefore, there is an urgent need for a preventative HIV-1 vaccine.

Although there is a great demand for a HIV-1 vaccine, no clinical trial candidate tested so far has provided effective long-lasting protection. HIV-1 is a genetically diverse pathogen which poses a significant challenge for vaccine design strategies. Through studying the HIV-1 specific B cell repertoire in chronically infected individuals, a class of rare antibodies that are capable of neutralizing diverse strains of virus were identified. These broadly neutralizing antibodies (bnAbs) were discovered to target specific regions (epitopes) of HIV-1's surface glycoprotein, envelope (Env). There are now efforts on designing vaccine immunogens capable of eliciting bnAbs in the context of HIV-1 vaccination.

The present disclosure also provides methods of developing, generating, and/or improving HIV vaccines.

In one aspect, disclosed herein is a method of preventing an infection from a human immunodeficiency virus (HIV), the method comprising administering to a subject an effective amount of a first composition comprising a commensal microbe antigen and an effective amount of a second composition comprising an HIV antigen.

In one aspect, disclosed herein is a method of boosting an immune response against a human immunodeficiency virus (HIV), the method comprising administering to a subject an effective amount of a first composition comprising a commensal microbe antigen and an effective amount of a second composition comprising an HIV antigen.

In some embodiments, the method of any preceding aspect boosts, increases, enhances, and/or optimizes an immune response against an HIV by 10%, 15%, 20%, 25%, 30%, 35%, 40%, 45%, 50%, 55%, 60%, 65%, 70%, 75%, 80%, 85%, 90%, 95%, 99%, 100%, or more relative to a control.

In some embodiments, the first composition is a booster for a vaccine regimen. In some embodiments, the second composition is a vaccine composition for the vaccine regimen. It should be noted that the vaccine regimen disclosed herein refers to a structured preventative plan or strategy for preventing infection and/or spread of an HIV virus. Generally, the vaccine regimen is designed and/or administered by a licensed medical practitioner. The vaccine regimen generally specifies the dosage, the vaccine scheduling, and the duration of time between booster(s) and vaccine. In some embodiments, the vaccine regimen comprises one or more compositions, wherein the compositions comprise either a booster, a vaccine, or any combination thereof. In some embodiments, the vaccine regimen comprises one or more therapeutic agents, including but not limited to adjuvants, preservatives, and the like. In some embodiments, the therapeutic regimen comprises any combination of compositions and therapeutic agents, such as for example the combination of a vaccine, a booster, and an adjuvant. In some embodiments, a therapeutic regimen comprises preventing a viral infection, including but not limited to HIV infections.

In some embodiments, the first composition is administered before the second composition. In some embodiments, the first composition is simultaneously administered with the second composition. In some embodiments, the first and/or second composition further comprises a pharmaceutically acceptable carrier including but not limited to any excipient, diluent, filler, salt, buffer, stabilizer, solubilizer, lipid, stabilizer, or other material well known in the art for use in pharmaceutical formulations.

The human intestinal microbiome is largely composed of bacteria, viruses, and fungi that directly impact host physiology through food digestion, the production of essential metabolites and protection from pathogens. Advances in genome sequencing technologies and bacterial culturing techniques have led to unprecedented insights into the composition and function of the intestinal microbial ecosystem. In healthy humans, the dominant bacteria phyla are Bacteroidetes, Firmicutes, Verrucomicrobia, Proteobacteria, and Actinobacteria, with factors such as diet, age, geography, and antibiotic use influencing the diversity of species present. Perturbation in intestinal microbiome diversity is associated with disease states such as inflammatory bowel disease and colorectal cancer.

The microbiome is colonized soon after birth, fluctuating during the first few years of life, until stabilizing in adulthood. The postnatal time period is a critical “window of opportunity” for the development and training of the immune system by microbial products. Accordingly, germ-free mice, that have never been exposed to microbes, display profound structural and functional immune system defects. In children, early exposure to antibiotics is associated with increased susceptibility to allergy and asthma and the development of wheezing and eczema in adulthood. Additionally, exposure to antibiotics in the first year of life is associated with increased risk for the development of inflammatory and metabolic diseases. These findings highlight the impact that carly life perturbations to the intestinal microbiome have on immune mediated disease. In summary, these insights demonstrate the profound role the intestinal microbiome has on host biology, with specific emphasis on immune system development, function, and homeostasis.

In some embodiments, the commensal microbe antigen is derived from an intestinal microbe. In some embodiments, the intestinal microbe comprises Limosilactobacilius reuteri (L. reuteri) or Bacteroides thetaiotaomicron (B. thetaiotaomicron).

In some embodiments, the commensal microbe antigen comprises at least 70%, 75%, 80%, 85%, 90%, 95%, 99%, or 100% sequence identity to SEQ ID NO: 7. In some embodiments, the commensal microbe antigen comprises at least 70%, 75%, 80%, 85%, 90%, 95%, 99%, or 100% sequence identity to SEQ ID NO: 59. In some embodiments, the commensal microbe antigen comprises Elongation Factor Tu (EF-Tu). In some embodiments, the EF-Tu comprises SEQ ID NO: 59, or a fragment thereof.

In one aspect, disclosed herein is a nucleic acid encoding an EF-Tu protein, wherein the nucleic acid comprises at least 70% sequence identity to SEQ ID NO: 60. In some embodiments, the nucleic acid comprises at least 70%, 75%, 80%, 85%, 90%, 95%, 99%, or 100% sequence identity to SEQ ID NO: 60.

The HIV envelope glycoprotein (Env) binds to cell surface-associated receptor (CD4) and coreceptor (CCR5 or CXCR4) by one of its two noncovalently associated subunits, gp120. The induced conformational changes activate the other subunit (gp41), which causes fusion of the viral with the plasma cell membranes resulting in delivery of the viral genome into the cell and initiation of the infection cycle. As the only HIV protein exposed to the environment, the Env is also a major immunogen to which neutralizing antibodies are directed, and a target which is relatively casy to access by inhibitors. A fundamental problem in the development of effective vaccines and inhibitors against HIV is the rapid generation of alterations at high levels of expression during long chronic infection and the resulting significant heterogeneity of the Env. The preservation of the Eny function as an entry mediator lends to the glycoprotein being an essential component for HIV vaccine development.

In some embodiments, the HIV antigen is derived from an HIV-1 virus. In some embodiments, the HIV antigen comprises an HIV glycoprotein, or a fragment thereof. In some embodiments, the HIV antigen comprises SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, or a fragment thereof.

In some embodiments, the HIV glycoprotein comprises a whole HIV envelope (Env) protein. In some embodiments, a fragment of the HIV glycoprotein comprises a CD4 binding (CD4bs), a CD4 induced site (CD4i), a VIV2 loop region (V1V2), a variable region 3 (V3), a membrane proximal external region (MPER), or a fusion peptide (FP).

In some embodiments, the method further comprises administering an adjuvant. In some embodiments, the adjuvant comprises a water-in-oil adjuvant. In some embodiments, the adjuvant comprises an aluminum salt (such as for example aluminum hydroxide, aluminum phosphate, and aluminum potassium sulfate), an oil emulsion, a monophosphoryl lipid A, cytosine phosphoguanine (CpG), phosphate salts, calcium salts, liposomes, toll-like receptor (TLR) agonists, and any combinations thereof.

As used herein, “vector” refers to any vehicle that carries a polynucleotide into a cell for the expression of the polynucleotide in the cell. The vector may be, for example, a plasmid, a virus, a phage particle, or a nanoparticle. Once transformed into a suitable host, the vector may replicate and function independently of the host genome, or may in some instances, integrate into the genome itself. In some embodiments, the vector is a DNA construct containing a DNA sequence which is operably linked to a suitable control sequence capable of effecting the expression of the DNA in a suitable host cell. Such control sequences can include a promoter to effect transcription, an optional operator sequence to control such transcription, a sequence encoding suitable mRNA ribosome binding sites, and sequences which control the termination of transcription and translation. In other embodiments, the vector comprises a lipid nanoparticle.

A plasmid or a viral vector can be capable of extrachromosomal replication or, optionally, can integrate into the host genome. As used herein, the term “integrated” used in reference to an expression vector (e.g., a plasmid or viral vector) means the expression vector, or a portion thereof, is incorporated (physically inserted or ligated) into the chromosomal DNA of a host cell. As used herein, a “viral vector” refers to a virus-like particle containing genetic material which can be introduced into a eukaryotic cell without causing substantial pathogenic effects to the eukaryotic cell. A wide range of viruses or viral vectors can be used for transduction but should be compatible with the cell type the virus or viral vector are transduced into (e.g., low toxicity, capability to enter cells). Suitable viruses and viral vectors include adenovirus, lentivirus, retrovirus, among others. In some embodiments, the expression vector encoding a chimeric polypeptide is a naked DNA or is comprised in a nanoparticle (e.g., liposomal vesicle, porous silicon nanoparticle, gold-DNA conjugate particle, polyethyleneimine polymer particle, cationic peptides, etc.).

In one aspect, disclosed herein is one or more vectors encoding the commensal microbe antigen or the HIV antigen of any preceding aspect. In some embodiments, the one or more vectors comprises at least 70%, 75%, 80%, 85%, 90%, 95%, 99%, or 100% sequence identity to SEQ ID NO: 60.

In some embodiments, the commensal microbe antigen and the HIV antigen enhances an immune response against the HIV relative to an HIV antigen alone. In some embodiments, the immune response comprises an increased HIV antibody response relative to a control. In some embodiments, the method prevents infection from the HIV.

The composition of any preceding aspect may be administered in such amounts, time, and route deemed necessary in order to achieve the desired result. The exact amount of composition of any preceding aspect will vary from subject to subject, depending on the species, age, and general condition of the subject, the severity of the HIV infection, the particular composition of any preceding aspect, its mode of administration, its mode of activity, and the like. The composition of any preceding aspect is preferably formulated in dosage unit form for ease of administration and uniformity of dosage. It will be understood, however, that the total daily usage of the composition of any preceding aspect will be decided by the attending physician within the scope of sound medical judgment. The specific effective dose level for any particular subject will depend upon a variety of factors including the virus being treated and the severity of the infection; the activity of the composition of any preceding aspect employed; the specific composition of any preceding aspect employed; the age, body weight, general health, sex and diet of the patient; the time of administration, route of administration, and rate of excretion of the specific composition of any preceding aspect employed; the duration of the treatment; drugs used in combination or coincidental with the specific composition of any preceding aspect employed; and like factors well known in the medical arts.

The composition of any preceding aspect may be administered by any route. In some embodiments, the composition of any preceding aspect is administered via a variety of routes, including oral, intravenous, intramuscular, intramedullary, intrathecal, subcutaneous, intraventricular, transdermal, interdermal, rectal, intraperitoneal, mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the composition of any preceding aspect (e.g., its stability in the environment of the body of the subject), the condition of the subject (e.g., whether the subject is able to tolerate the chosen route of administration), etc.

The exact amount of composition of any preceding aspect required to achieve a effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

In some embodiments, the composition of any preceding aspect can be prepared as a “concentrate”, e.g. in a storage container of a premeasure volume and/or a predetermined amount ready for dilution, or in a soluble capsule ready for addition to a specified volume of water, saline, alcohol, hydrogen peroxide, or other diluent.

In some embodiments, the composition of any preceding aspect is administered 1, 2, 3, or more times. In some embodiments, the first and second compositions of any preceding aspect are administered simultaneously. In some embodiments, the first and second compositions of any preceding aspect are administered on the same day. In some embodiments, the first and second compositions of any preceding aspect are administered at least one day apart from each other. In some embodiments, the first and second compositions of any preceding aspect are administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365 days apart from each other.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Priming the Anti-HIV Antibody Response with an Antigen from Commensal Bacteria

The human immunodeficiency virus uses its envelope (Env) protein to bind and enter host cells. Env is located on the surface of the virus and is a trimer composed of glycoprotein (gp) 120 and gp41 heterodimers. Importantly, Env is the sole target of neutralizing antibodies (Nabs), and it is thought for a vaccine candidate to be successful it must elicit this type of antibody response. To date, eight large vaccine efficacy trials have been completed with only one, RV144, showing modest efficacy. Although 60% vaccine efficacy was seen at 12months, this waned to 31.2% after 40 months. In efforts to improve upon the 30% efficacy seen in RV144, an analogous trial HVTN 702 recently took place in South Africa. Unfortunately, the study was ended carly as no significant effect of the vaccine regimen on the prevention of HIV-1 infection was seen. The repeated failure of vaccine candidates has led to an intense search for explanations for the heterogeneity of immune responses seen among clinical trial participants.

One host-specific factor that has recently been implicated in influencing the immune response to vaccination is the intestinal microbiome. Initial work done in germ-free or antibiotic-treated mice showed a significant decrease in antigen-specific IgG and IgM levels following vaccination with a seasonal flu vaccine. A similar trend was seen in humans who were given antibiotics prior to receiving the seasonal flu vaccine. In this study, participants with low pre-existing immunity to influenza had a decreased influenza-specific IgGI and IgA response when administered antibiotics prior to vaccination. With respect to HIV-1, particular HIV-1 monoclonal antibodies (mAbs), have been shown to be cross-reactive with gut commensal antigens. These antibodies originate from both infected and vaccinated individuals, they tend to be non-neutralizing, directed towards the gp41 region of Env and cross-reactive with the intestinal microbiome. Additional data from multiple HIV vaccine clinical trials found that particular gut commensal bacterial correlate with anti-HIV-1 envelope (Env) IgG levels. The studies contemplated that the gut microbiome may be shaping the HIV-1 antibody response.

Herein, the present disclosure seeks to better understand the role of the intestinal microbiome on the HIV-1 antibody response. The cross-reactivity of HIV-1 monoclonal antibodies with lysate from three well-studied, commensal bacterium, Limosilactobacillus reuteri (L. reuteri), Bacteroides thetaiotaomicron (B. thetaiotaomicron) and Akkermansia muciniphila (A. muciniphila) was examined. The HIV-1 antibodies, CH58 and Z13e1, were found to be cross-reactive with commensal bacteria lysate. Using immunoprecipitation and mass spectrometry analysis, binding partners of CH58 were identified. Follow-up analysis shows the binding of CH58 to L. reuteri lysate is not due to general polyreactivity of the antibody and is likely the result of an antigen specific interaction. Data supports elongation factor Tu (EF-Tu), a conserved and abundant protein found in bacteria, as a binding partner of antibody CH58. Additionally, to further understand the role of commensal antigens such as EF-Tu, in influencing the HIV-1 antibody response, guinea pigs were immunized with recombinant EF-Tu protein, followed by immunization with HIV-1 Env protein, BG505 DS-SOSIP, and monitored the development of antigen-specific IgG antibody responses. Boosting with EF-Tu protein, compared to placebo boost, increases anti-Env antibody titers, and influences the immunogenic region targeted. These findings have important implications for HIV-1 vaccine development, as preexisting anti-commensal antibody responses influence the development of a protective HIV-1 antibody response.

### Cross Reactivity Profiles of HIV-1 Monoclonal Antibodies Against Gut Commensal Bacteria

With the goal of testing the cross-reactivity of HIV-1 monoclonal antibodies to intestinal microbiota, a screening through western blot analysis of lysate from the commensal microbe Limosilactobacillus reuteri (L. reuteri) was performed against a diverse panel of well-studied, HIV-1 specific monoclonal antibodies. The panel included twelve HIV-1 specific mAbs targeting various sites on the HIV-1 envelope (Env) protein including: the CD4 binding site (CD4bs), the CD4 induced site (CD4i), the VIV2 loop region (V1V2), the variable region 3 (V3), the membrane proximal external region (MPER) and the fusion peptide (FP). Ten of the twelve antibodies display neutralizing activity, with two of the twelve displaying weak to no neutralizing activity. One of the non-neutralizing antibodies in the panel, CH58, displays antibody-dependent cell-mediated cytotoxicity (ADCC) activity and is the only mAb to be isolated from a vaccine trail participant.

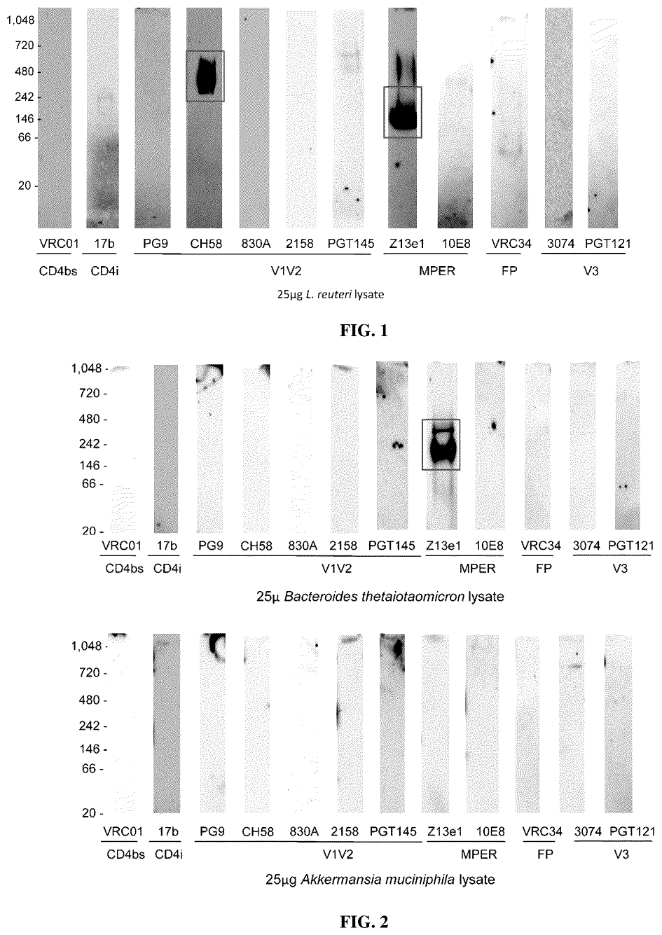

The rest of the twelve antibodies were isolated from HIV-infected individuals (Table 1). Of the antibodies tested, two, CH58 and Z13e1, showed strong signal through western blot analysis to L. reuteri lysate (FIG. 1).

To further probe the cross-reactivity of the HIV-1 antibody panel, screening of lysate from two additional commensal bacteria, Bacteroides thetaiotaomicron (B. thetaiotaomicron) and Akkermansia muciniphila (A. muciniphila), was performed using western blot analysis. Of the twelve antibodies, none bound lysate from A. muciniphila, and only one, Z13e1, bound lysate from B. thetaiotaomicron (FIG. 2). Together these data show few HIV-1 monoclonal antibodies included in the panel are cross-reactive with antigens from commensal bacteria.

### HIV-1 Antibodies Show Diverse Levels of Polyreactivity

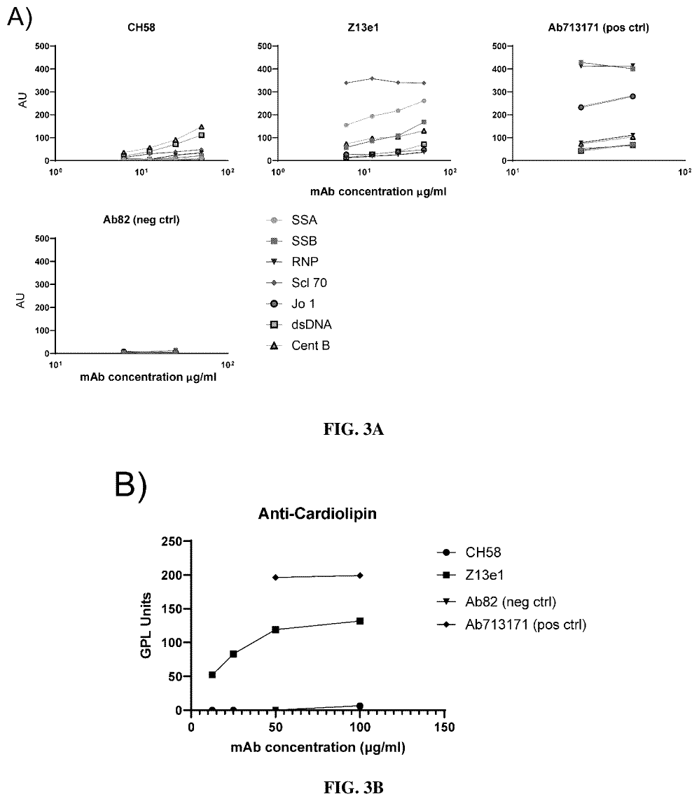

To assess the nature of the HIV-1 antibody cross-reactivity seen, the reactivity of antibodies, CH58 and Z13e1, to a panel of self-antigens was measured using the Luminex AtheNA Multianalyte ANA assay (FIG. 3A). Similarly to what has been determined in previous studies, antibody Z13e1 displayed high levels of autoreactivity for 2/8 autoantigens tested (FIG. 3A). CH58 displayed no autoreactivity for all of the 8 autoantigens tested (FIG. 3A). In addition, autoreactivity was further tested by measuring anti-cardiolipin reactivity for both mAbs. Z13e1 displayed positive binding for cardiolipin antigen whereas CH58 displayed negligible binding profiles (FIG. 3B). Together these data show that only mAb Z13e1 showed binding in autoreactivity assays, showing that the bacterial cross-reactive phenotype seen by CH58 may not be explained by non-specific antigen interactions.

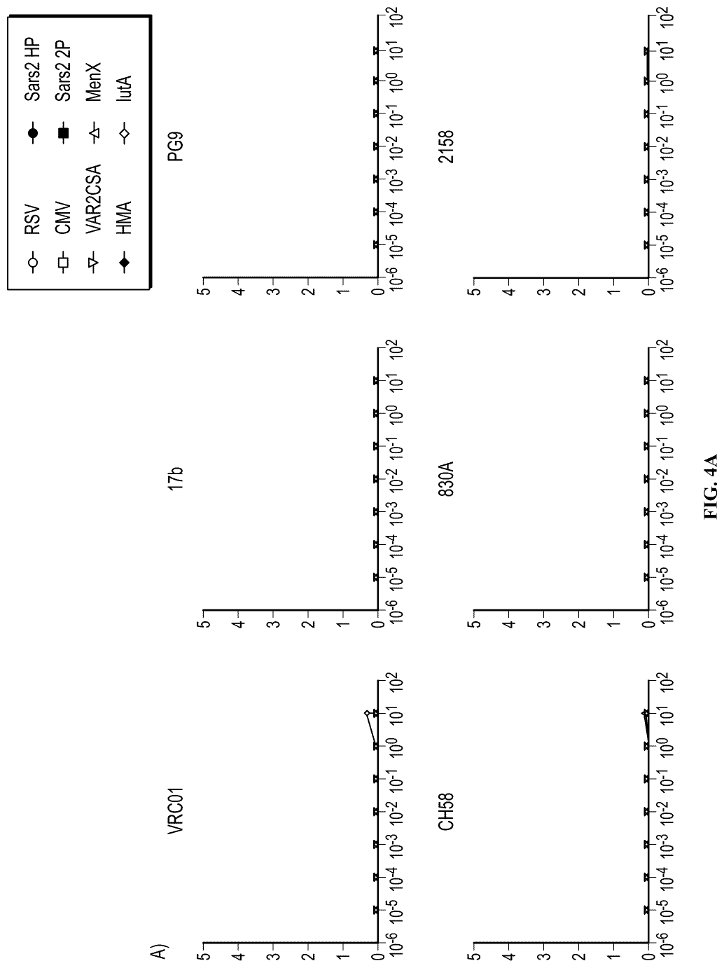

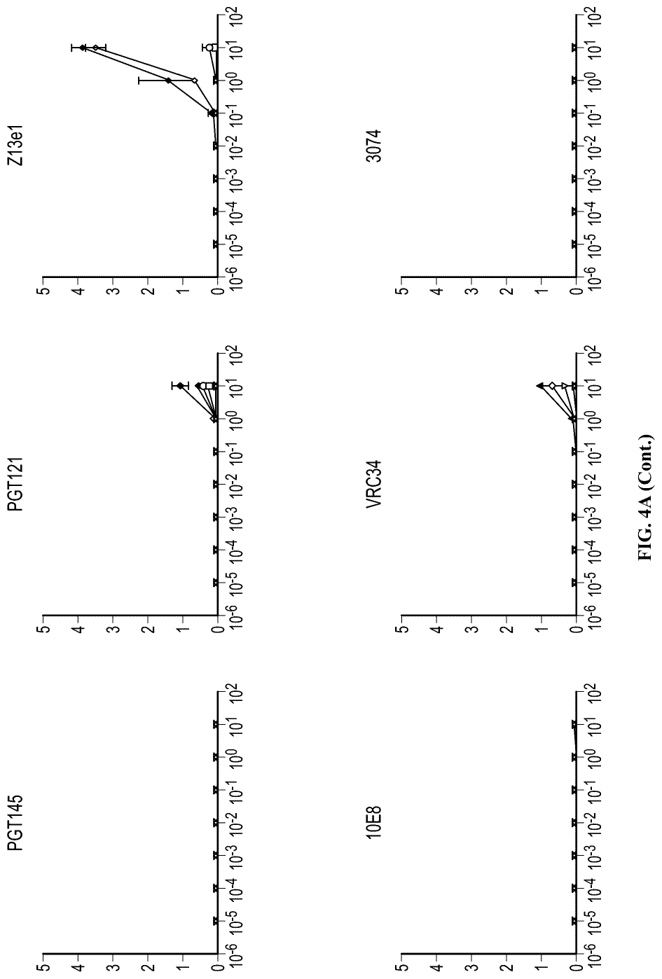

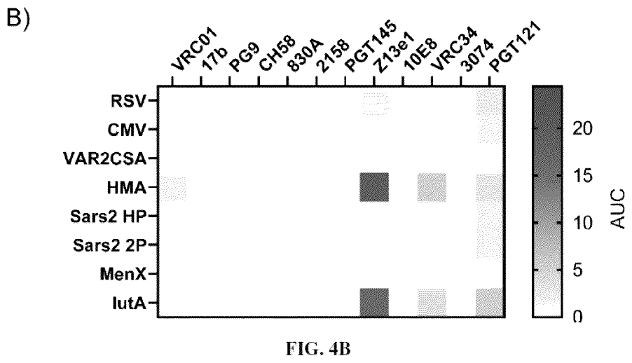

In addition to autoreactivity, the present disclosure also tests the HIV-1 mAb panel for cross-reactivity to a diverse set of bacterial, viral, and parasitic antigens. Included in the panel were four viral proteins, respiratory syncytial virus postfusion F (RSV F), human cytomegalovirus glycoprotein B (CMV), SARS-COV-2 spike S-2P (Sars2 2P), and SARS-COV-2 spike HexaPro (Sars2 HP). One parasitic protein, malaria surface antigen (VAR2CSA). In addition, the panel contained three bacterial proteins, Escherichia coli (E. coli) heme acquisition protein (HMA), Meningococcal factor H binding protein (MenX) and E.coli ferric acrobactin receptor (IutA). Of the twelve HIV-1 mAbs, only Z13e1 displayed high levels of polyreactive binding to antigens in the panel (FIGS. 4A and 4B). All other mAbs displayed low to no cross-reactivity to any of the antigens tested. This data is in agreement with the autoreactivity data and lends evidence to support that the cross-reactive phenotype displayed by mAb CH58 cannot be explained by non-specific antigen interactions.

### L. reuteri Antigen Elongation Factor Tu Identified as an Antigen Target of CH58

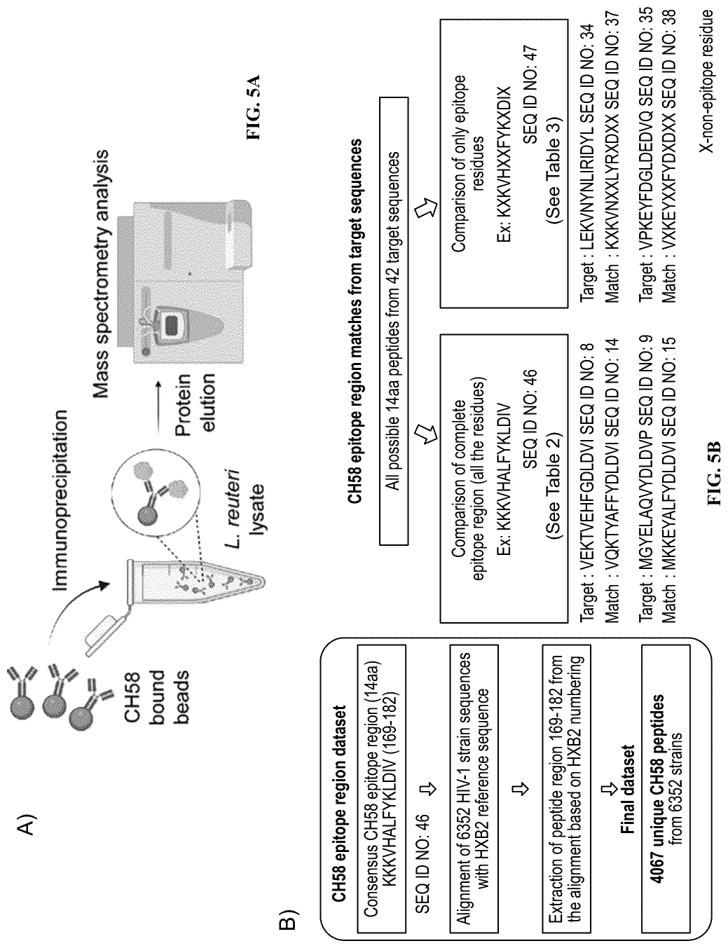

To identify potential binding partners for antibody CH58 present in L. reuteri lysate, immunoprecipitation (IP) was performed followed by subsequent mass spectrometry analysis (FIG. 5A). CH58 was covalently coupled to magnetic beads and incubated with L. reuteri lysate. The IP clute was separated by protein gel electrophoresis and subjected to mass spectrometry analysis. The resulting hits identified through mass spectrometry were then prioritized based off sequence alignment with CH58′s known epitope region KKKVHALFYKLDIV (SEQ ID NO: 47) (FIG. 5B). Through this process, several proteins were identified, including Elongation Factor Tu (EF-Tu), as binding partners of CH58.

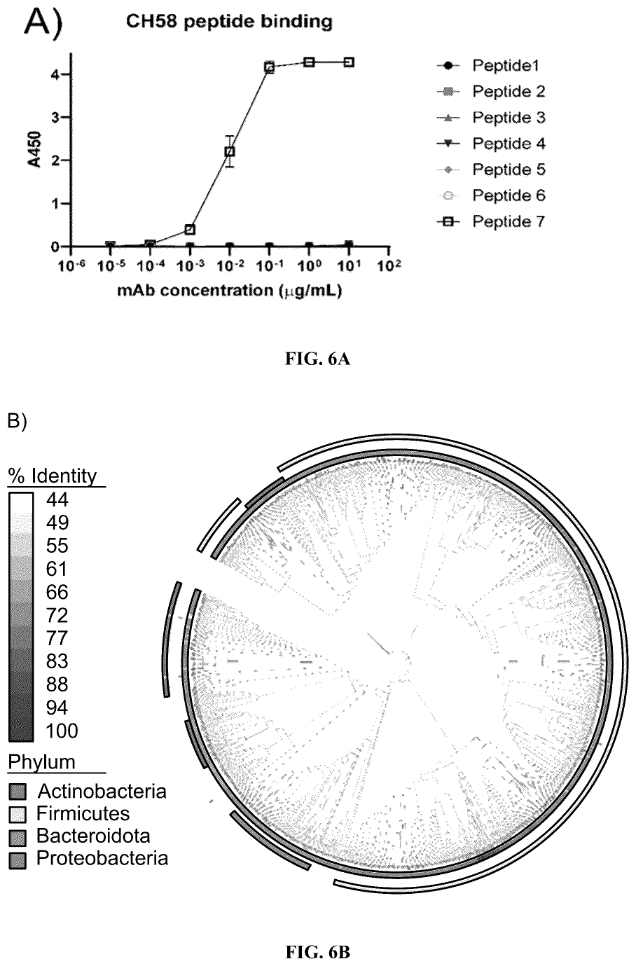

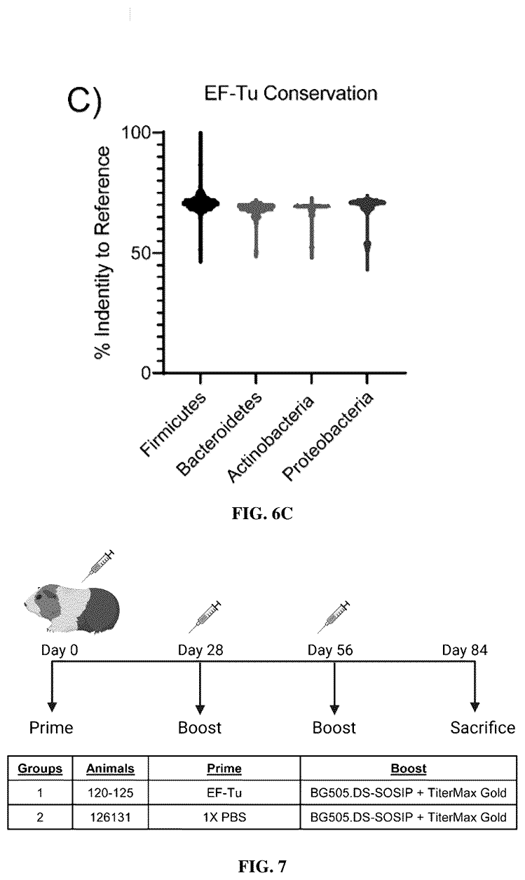

CH58 binds a linear epitope on Env, therefore, it was contemplated that CH58 would similarly bind a linear epitope on EF-Tu. Six linear EF-Tu peptides were tested for binding to CH58 by ELISA (Table 4 and FIG. 6A). The six peptides tested, EF-Tu_1 through 6, were identified from the computational analysis of the immunoprecipitation mass spectrometry hits (FIG. 6B). This analysis revealed six peptide regions from L. reuteri 's EF-Tu protein that matched, with 78% sequence similarity, to the CH58 epitope region of at least one of the 6352 HIV-1 strains queried. Peptide 7, EF-Tu_7, was a positive control peptide of the known CH58 epitope on Env, ELRDKKQKVHALFKLDIV (SEQ ID NO: 7). CH58 displayed no binding to any of the EF-Tu peptides tested. Under the conditions tested, binding of CH58 to any of the tested peptides was not observed, showing that CH58's epitope on EF-Tu is non-linear (FIG. 6A).

Next, the present disclosure determined the sequence conservation of EF-Tu protein among commensal bacteria. Utilizing the Unified Human Gastrointestinal Genome (UHGG) catalogue, the percent sequence conservation of EF-Tu among the genomes of 4,644 intestinal microbe species representatives was examined. By aligning L. reuteri 's EF-Tu amino acid sequence against the UHGG species representatives, EF-Tu was determined to be highly conserved among members of the intestinal microbiome (FIG. 6B and FIG. 6C). The conservation of EF-Tu postulates whether naïve B cells could be primed by conserved commensal antigens and later go on to recognize HIV-1 antigen.

### Bacterial Antigen Prime Influences HIV-1 Specific Antibody Responses

EF-Tu was identified as being bound by HIV-1 antibody CH58 and in agreement with previous data, EF-Tu is highly conserved among members of the intestinal microbiome, because of this the present disclosure directly investigates its effect on HIV-1 vaccine response. To accomplish this, Hartley guinea pigs were immunized with recombinant EF-Tu protein, followed by two booster immunizations with BG505 DS-SOSIP Env trimer (FIG. 7). For comparison, a control group was included in the study that received PBS as a prime followed by two booster immunizations with BG505 DS-SOSIP Env trimer (FIG. 7). TiterMax Gold was used as an adjuvant in each boost step. BG505 DS-SOSIP was chosen as an immunogen because it contains stabilizing mutations that allow the host's immune system to recognize the trimer in a way that mimics how the Env trimer would be displayed on HIV-1's surface, making these immunogens a prime candidate for vaccine development.

### Design for Animal Immunizations

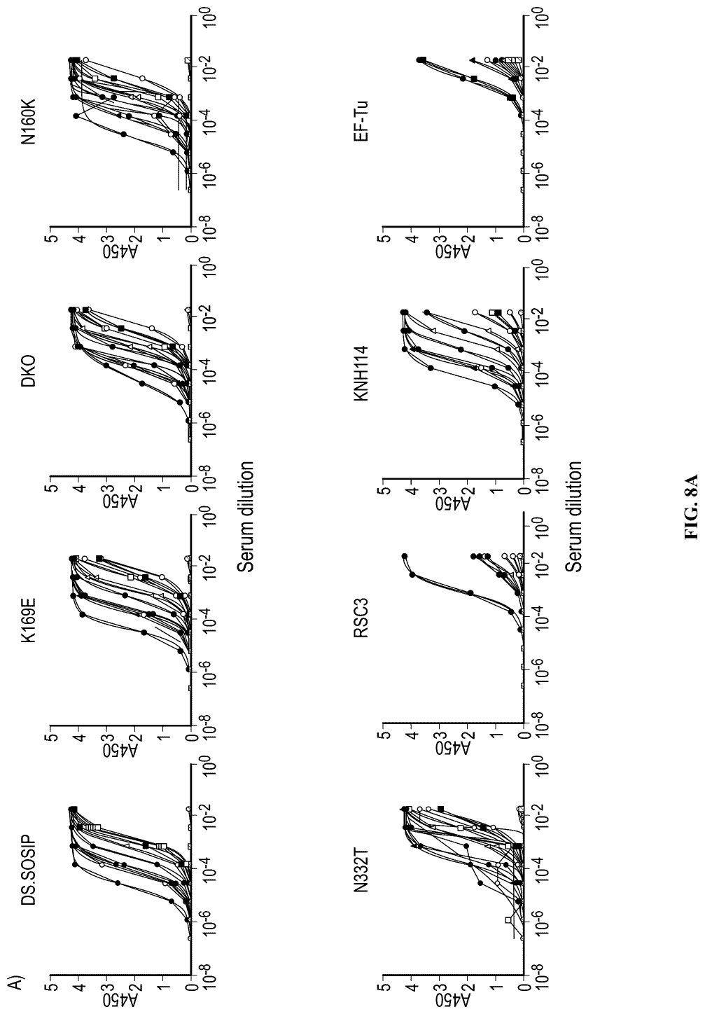

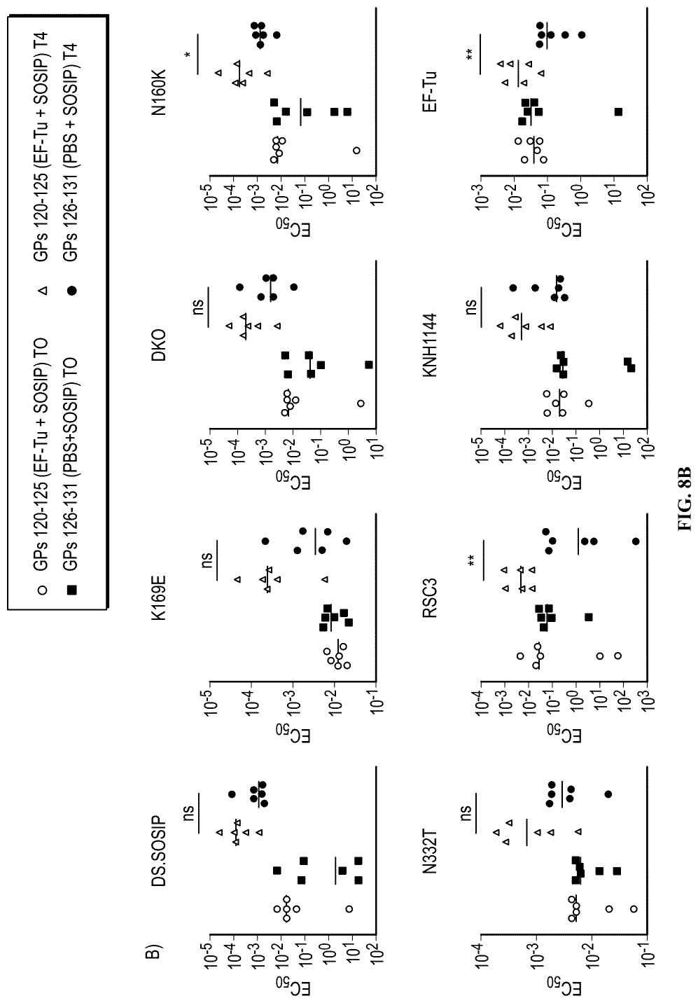

The present disclosure seeks to compare the elicitation of antigen-specific IgG antibody responses between each immunization group. The serological antibody response to each immunogen and a panel of HIV-1 Env proteins was measured using ELISA (FIG. 8A). Scrum dilution titers were also quantified with a 50% effective concentration (EC₅₀) of EF-Tu and Env specific antibodies (FIG. 8B). Interestingly, compared to the PBS prime group, animals boosted with EF-Tu prior to immunization with Env protein, elicited a more robust antibody response to 2 out of the 5 HIV-1 antigens tested (FIG. 8B). Animals in the group that were not immunized with EF-Tu protein did not elicit a robust EF-Tu antibody response (FIGS. 8A and 8B). Intriguingly, EF-Tu primed animals elicited a greater antibody response to HIV-1 antigen RSC3 (Recombinant re-stabilized core 3 WT) which was designed to mimic the neutralizing face of

Env's CD4bs and has been used to isolate several well characterized neutralizing CD4bs directed antibodies. Taken together, these findings show that priming an HIV-1 vaccine regimen with a non-HIV-1 antigen, specifically an antigen from a commensal bacteria, leads to the elicitation of a stronger HIV-1 antibody response and shift the response towards specific antigenic epitopes of interest.

### Discussion

HIV-1 vaccine development has been faced with many hurdles, and understanding host factors that influence vaccine efficacy is critical to the development of an effective vaccine candidate. This is also true of many other infectious diseases that have been resistant to modern vaccine efforts. The intestinal microbiome has recently been implicated in immune system development and vaccine responsiveness. However, the extent of this effect on HIV-1 vaccine response, the particular strains of bacteria involved, and the antigen-specific nature of this effect have not been fully investigated. Herein, the cross-reactive profiles of a panel of well-studied HIV-1 antibodies were examined against three diverse strains of commensal bacteria. Additionally, the HIV-1 antigen-specific antibody response in animals boosted was profiled with the commensal antigen EF-Tu. HIV-1 antibody CH58 cross-reacts with the intestinal microbe, L. reuteri, in an antigen-specific manner. Additionally, it was found that immunization with EF-Tu prior to immunization with HIV-1 Env protein leads to an increased anti-Env antibody response and shifts the anti-Env epitope landscape.

In the initial phases of HIV-1 infection, anti-gp41 antibodies dominate the antibody response, and some of these antibodies have been shown to be cross-reactive with the microbiome. Additionally, in follow up studies of the failed vaccine trial, HVTN 505, the antibody response elicited by the vaccine immunogen was primarily non-neutralizing and cross-reactive with the intestinal microbiome. Herein, it was also identified that pre-vaccination intestinal microbome-gp41 cross-reactive B cells, therefore confirming the existence of a pre-vaccination pool of Env-Intestinal microbiome cross-reactive B cells. With this, there was further investigation of the cross-reactive nature of the HIV-1 antibody response.

Through an HIV-1 antibody screen, antibody CH58 displayed binding to the commensal bacteria, L. reuteri. CH58 was isolated from a participant in the only HIV-1 vaccine clinical trial to show any vaccine efficacy, RV144. CH58 was found to target the VIV2 region of Env and follow-up analysis of this trial found that antibodies targeting this region were associated with protection from HIV-1 infection. Similar conclusions have been found in non-human primate studies, where protection, control, and/or delayed infection with SIV or SHIV were correlated with strong antibody responses to the VIV2 domain on Env. Previous studies investigating HIV-1 antibody cross-reactivity have found non-neutralizing, gp41 reactive antibodies primarily to be cross-reactive with the intestinal microbiome. Antibody CH58 is non-neutralizing, however it mediates ADCC against HIV-infected CD4⁺ T cells. Interestingly, ADCC activity, not neutralization, was another correlate of protection in the RV144 trial and lysis of infected cells is a proposed mechanism of protection against HIV-1 infection. The finding herein of antibody CH58 displaying cross-reactivity to L. reuteri, shows that antibodies targeting protective immunogenic regions, such as VIV2, and that initiate protective effector functions, can also cross-react with the intestinal microbiome.

Under physiologic conditions, antigen-specific B cell responses are directed towards intestinal microbes and up to 80% of antibody-secreting B cells are located in the gut mucosa. This fact highlights the magnitude of the gut microbiome on shaping the B cell repertoire. Previous studies have discovered associations of different gut taxa with response to various vaccine regimens, including HIV. Additionally, to follow up on their discovery of gp41 commensal bacterial antigen antibodies, Trama and colleagues, identified E. coli RNA polymerase as one commensal antigen recognized by HIV-1 gp41 antibodies (Trama AM, Moody MA, Alam SM, Jaeger FH, Lockwood B, Parks R, et al. HIV-1 envelope gp41 antibodies can originate from terminal ileum B cells that share cross-reactivity with commensal bacteria. Cell Host Microbe. 2014; 16(2):215-26). The present disclosure differs from these studies by seeking to probe the commensal bacterial antigen landscape capable of priming anti-HIV-1 B cells. For this three diverse, well studied, members of the intestinal flora were chosen to screen against, L. reuteri, B. thetaiotaomicron and A. muciniphila. The finding of only L. reuteri, showing strong levels of cross-reactivity to only one antibody, CH58, shows the cross-reactivity of mature HIV-1 mAbs to commensal antigens, is rare. Nevertheless, the finding of EF-Tu, being a binding partner of CH58 shows that, B cells against conserved and abundant bacterial antigens, are capable of responding to HIV-1 Env protein. Interestingly, while not a highly abundant species found in the gut, such as B. thetaiotaomicron and A. muciniphila, L. reuteri, is considered an earlier colonizer of the gastrointestinal tract, being one of the first species infants are colonized with through transfer from the mother after birth. The finding of HIV-1 antibody CH58 cross-reacting with an antigen from L. reuteri, taken together with previous literature showing antigen-specific B cell repertoires are developed shortly after birth by commensal microbes, shows that HIV-1 commensal cross-reactive B cells can be developed early in life (Wesemann DR, Portuguese AJ, Meyers RM, Gallagher MP, Cluff-Jones K, Magee JM, et al. Microbial colonization influences early B-lineage development in the gut lamina propria. Nature. 2013;501(7465): 112-5).

The final finding of EF-Tu immunization, increasing anti-Env antibody response levels and potentially steering the antibody response, raises interesting questions about the role of anti-commensal B cells in the response to HIV-1 vaccination. This is the first study to show prime with a non-HIV-1 antigen, leading to an increased anti-Env response. Eliciting a high titer, antibody response to a vaccine immunogen is a major goal of vaccine clinical trials. One promising finding of the animal study is the EF-Tu prime group elicited a more robust antibody response to RSC3 protein, a protein that preferentially reacts with broadly neutralizing CD4bs antibodies. This data shows that priming HIV-1 Env vaccination with a non-HIV-1, commensal antigen, can boost the HIV-1 neutralizing response in these animals and redirect the serum antibody response to epitopes on Env.

### Methods and Materials

### Measurement of Bacterial Lysate and Antigen Binding by Western Blot

Limosilactobacillus reuteri (strain 32035) was purchased from The Leibniz Institute DSMZ-German Collection of Microorganisms and Cell Cultures. The bacteria were grown anacrobically in MRS broth to an OD of 7.56. Akkermansia muciniphila, YL44, was purchased from the DSMZ. It was streaked onto Brain Heart Infusion (BHI) agar (BD Difco #211059) supplemented with 5% (v/v) defibrinated sheep blood and cultured anaerobically for 3 days. Single colonies were inoculated into BHI broth containing 4 g/L pig-derived mucin and grown for 7 days anacrobically. B. thetaiotaomicron VPI 5482 Δtdk was a gift from Dr. Eric Martens. It was cultured on Brain Heart Infusion (BHI) agar (BD Difco #211059) supplemented with 5% (v/v) defibrinated sheep blood for 1 day and then colonies inoculated into BHI broth. Cultures were grown anaerobically at 37° C. for 3 days. All cultures were then pelleted and resuspended in 5 mL of 1× phosphate buffered saline (1×PBS) and added to glass beads and vortexed for 10 minutes.

The lysate was spun down and the lysate supernatant was used for downstream analysis. Protein concentration was determined by the Pierce BCA Protein Assay Kit (ThermoFisher).

For native western blot analysis, 25 μg of bacterial lysate was run on Novex Tris-Glycine gels (ThermoFisher) for 35-55minutes at 225V under non-reducing conditions. Samples were prepared with native sample buffer (Bio-Rad). For non-native western blotting bacteria lysate was run on Mini-Protean gels (Bio-Rad) for 30-40 minutes at 200V. Samples were reduced in 4× laemmli sample buffer (Bio-Rad) with β-mercaptoethanol added. Proteins were then transferred to a nitrocellulose membrane using Invitrogen's iBlot2 transfer system. Primary and secondary antibody wash steps were performed with Invitrogen's iBind Western System. Primary antibodies were diluted to 10 μg/ml and secondary antibody, goat anti-human IgG antibody-HRP (Jackson ImmunoResearch) was diluted 1:2000. SuperSignal West Pico PLUS Chemilumiescent (ThermoFisher) substrate was used to develop the membrane and signal was detected using The Digital ChemiDoc MP (Bio-Rad).

### Enzyme-Linked Immunosorbent Assay (ELISA)

To assess antibody binding to antigen, recombinant protein ELISAs were performed. For animal serum analysis, antigen was plated at 2 μg/mL on Immulon 2HB overnight at 4° C. The next day plates were washed three times with PBS supplemented with 0.05% Tween-20 (PBS-T) and blocked with 5% NFDM in PBS-T. Plates were incubated for one hour at room temperature (RT) and washed three times with PBS-T. Scrum samples were diluted in 1% NFDM in PBS-T, starting at a 1:50 dilution with a serial 1:5 dilution and then added to the plate. The plates were incubated at RT for one hour and then washed three times in PBS-T. The secondary antibody, goat anti-Guinea Pig IgG conjugated to peroxidase (Thermo Fisher), was added at 1:10,000 dilution in 1% NFDM in PBS-T to the plates, which were incubated for one hour at RT. Plates were washed three times with PBS-T and then developed by adding TMB substrate (Thermo Fisher) to each well. The plates were incubated at room temperature for ten minutes, and then IN sulfuric acid was added to stop the reaction. Plates were read at 450 nm. Data is represented as mean±SEM.

### Antibody Expression and Purification

For antibodies VRC01, 17b, PG9, CH58, 830A, 2158, PGT145, 10E8, VRC34, 30-3074 and PGT121 variable gene sequences were inserted into custom plasmids encoding the heavy chain IgGI constant region and the corresponding lamda or kappa light chain region (pTwist 314 CMV BetaGlobin WPRE Neo vector, Twist Bioscience). Antibodies were expressed in Expi293F mammalian cells (Thermo Fisher) with PEI transfection reagent and cultured for 5-7 days in FreeStyle F17 expression Medium supplemented with 10% Pluronic acid and 20% glutamine. Cells were maintained at 37° C. with 8% CO2 saturation while shaking. After 5-7 days cells were spun down and supernatant was harvested. Supernatant was run over a protein A affinity column. The column was washed with 1×PBS and the protein was eluted with 100 mM Glycine HCI at 2.7 pH directly into a 1:10 volume of IM Tris-HCl pH 8.0. Eluted antibodies were buffer exchanged using Amicon Ultra-centrifugal filter units into 1×PBS and stored for future use. Antibody Z13e1 was obtained through the NIH HIV Reagent Program, Division of AIDS, NIAID, NIH: Anti-Human Immunodeficiency Virus 1 gp41 Monoclonal Antibody, ARP-11557, contributed by Dr. Michael Zwick.

### Immunoprecipitation

HIV-1 antibody CH58 that bound to L. reuteri lysate through western blot and ELISA was used for Immunoprecipitation (IP). The antibody was coupled to the surface of magnetic Dynabeads M-270 Epoxy beads per manufacturers instruction (ThermoFisher). Briefly, 100 μg of CH58 was incubated with 5 mg of Dynabeads M-270 Epoxy beads and incubated for 37° C. while shaking overnight. The CH58 bound beads were then collected using a magnet and the supernatant removed. The beads were then washed. This was repeated 4 times and the beads were stored at 4° C. until use. L. reuteri lysate was prepared as described above, 1 mg of lysate was mixed with CH58 bound beads and incubated for 2 hours at RT while mixing. The beads were then collected using a magnet and the supernatant removed. The beads were then washed with PBS-T. This process was repeated twice. Protein bound to CH58 was then eluted using 20 mM glycine pH 2.0. The IP elute was then separated using SDS-PAGE gel electrophoresis and stained with SimplyBlue SafeStain (Thermo Scientific). The prominent bands were cut and sent for mass spectrometry analysis.

### Mass Spectrometry Analysis

Proteins were identified by first digesting them into tryptic peptides via S-trap (Protifi-S-Trap-ProtiFi). Resulting peptides were analyzed by data dependent LC-MS/MS. Briefly, peptides were autosampled onto a 200 mm by 0.1 mm (Jupiter 3 micron, 300A), self-packed analytical column coupled directly to an QE+ orbitrap mass spectrometer (ThermoFisher) using a nanoelectrospray source and resolved using an aqueous to organic gradient. Both the intact masses (MS) and fragmentation patterns (MS/MS) of the peptides were collected in a data dependent manner utilizing dynamic exclusion to maximize depth of coverage. Resulting peptide MS/MS spectral data were searched, using SEQUEST (150) . Peptide spectral matches (PSMs) collated, filtered, and compared using Scaffold (Proteome Software).

### CH58 Epitope Matching from Mass Spectrometry Hits

CH58 epitope matches to mass spectrometry target sequences were retrieved in two steps using in-house developed perl scripts: 1) retrieving CH58 epitope regions from all HIV-1 strains, 2) scanning the target sequence (using the regions retrieved in the first step) to find similar epitope matches. To extract CH58 epitope matches, the alignment of 6352 HIV-1 strains were retrieved from the Los Alamos National Laboratory (LANL) database. From the alignment, CH58 epitopc regions (from amino acid residue 169 to 182) from all the HIV-1 strains were extracted based on HXB2 reference sequence numbering. After removing regions with gaps (-) and unknown residues (X), 4067 unique 14aa peptides were collected from 6352 HIV-1 strains (dataset1). To scan the target sequence for the epitope matches, all the possible 14 aa peptides were extracted from the target sequence (dataset2). Then, the epitope matches were found using two approaches. In the first approach, all the peptides from dataset1 compared with all the peptides in the dataset2 and the amino acid identity for each pair computed by all the residues positions (169-182 or 14 aa) from the peptides. In the second approach, instead of all only the 8 epitope residue positions (169, 171-173, 176-178, 180) were considered to compute amino acid identity between peptides.

### Elongation Factor Tu Sequence Analysis

Amino acid sequences from 4,744 species representatives of prokaryotic genomes from the human gut microbiome were downloaded from the Unified Human Gastrointestinal Genome (UHGG) collection. The data set was trimmed by first pulling only sequences with annotated EF-Tu genes, then any EF-Tu protein sequence with less than 350 amino acids was excluded. The amino acid sequence of EF-Tu from L. reuteri (strain JCM 1112) was then compared to the remaining sequences to calculate % identity. From this list, a genus representative was chosen with the highest percent identity match to L. reuteri's EF-Tu sequence. A phylogenetic tree was created using iTOL v6. Interactive Tree Of Life.

### Elongation factor Tu Expression and Characterization

Cloning: Genomic DNA was isolated from L. reuteri using the Purelink Genomic DNA Mini Kit (Invitrogen) and the isolated genomic DNA was quantified by nanodrop. PCR was used to amplify the EF-Tu from the gDNA. Forward and reverse primers were designed to include the kit vector-specific sequences on the 5′-end, and EF-Tu-specific sequences on the 3′-end. Primers were synthesized by IDT. Gradient PCR of the EF-TU gene was performed to determine the optimal annealing temperature using the Phusion High Fidelity PCR Master Mix with HF buffer (NEB). PCR reactions were with 10 ng of gDNA, and a final primer concentration of 0.5 uM. Reactions containing DMSO to a final concentration of 3% were also included. PCR thermocycler conditions were initial denaturation at 98° C. for 30 sec, denaturation at 98° C. for 10 sec, annealing range at 50-72° C. for 30 sec and extension at 72° C. for 30 sec. Annealing and extension steps were repeated 30 times. The final extension temperature was 72° C. for 10 min. PCR reactions were visualized on a 1% SYBR Safe agarose gel in 1×TAE (Tris-Acetate EDTA) buffer. PCR of the EF-Tu gene was repeated as above using 25 ng of gDNA with an annealing temperature of 68° C. and the addition of DMSO to a final concentration of 3%. The 1224 bp PCR product was excised from a 1% SYBR Safe agarose gel and the DNA extracted using the Qiaquick Gel Extraction kit (Qiagen). The A260/A280 was determined using a nanodrop. Directional LIC (Ligation Independent Cloning) was performed using the aLICator Ligation Independent Cloning and Expression Kit (Thermo Scientific). The cloning reaction was a 2-step process. The first step was the generation of 5′ and 3′ overhangs with 1×LIC buffer, 0.1 pmol gel extracted EF-Tu and 0.1U final concentration T4 DNA polymerase. The reaction was incubated at room temperature for 5 min. The second step, the annealing reaction, included the addition of 60 ng of pLATE51 vector to the 5′/3′ overhang mixture and incubating at room temperature for 5 min. The reaction was stopped with 0.6 ul of 0.5 M EDTA. One Shot TOP10 chemically competent cells (Invitrogen) were transformed with 2.5 ul of the annealing mixture. Briefly, the cells and mixture were incubated on ice for 15 min followed by heat shocking the cells at 42° C. for 30 sec and then placing on ice. Two hundred and fifty ul of SOC was added and the cell mixture incubated at 37° C. for 1 h at 225 rpm. One hundred ul of the SOC mixture was plated onto and LB agar containing 100 ug/ml ampicillin. Plasmid DNA was prepared from 5 colonies using the QiaPrep Spin Mini Prep kit (Qiagen) and PCR amplified using primers provided in the aLICator Ligation Independent Cloning and Expression Kit to determine the presence of the EF-Tu insert. Plasmid DNA containing the insert was identified and used to transform BL21 Star (DE3) chemically competent cells (Invitrogen). Transformation was with 5 ng of plasmid DNA and method was as previously described above. Colony PCR was performed on 5 transformants to identify the EF-TU insert. Glycerol stocks were prepared from two clones containing the plasmid with the EF-Tu insert.

Protein Expression and Purification: EF-Tu/pLATE51/BL21 Star (DE3) glycerol stock was plated out on LB agar containing 100 ug/ml ampicillin. One colony was cultured in LB broth containing 100 ug/ml ampicillin and 1% glucose and incubated at 37° C. at 225 rpm overnight. The following day, 2 ml of the overnight culture was added to 100 ml LB broth containing 100 ug/ml ampicillin and 1% glucose and incubated at 37° C., 225 rpm until an OD600 0.5-0.8. The culture was induced with 1M IPTG (isopropyl ß-D-1-thiogalactopyranoside) to a final concentration of 1 mM, and incubated at 37° C., 225 rpm for 4 h. A non-induced culture was also incubated. Cells were pelleted by centrifugation for 20 min at 10,000 rpm and stored at −20° C. overnight. Bacterial pellets were thawed on ice and then resuspended in lysis buffer (50 mM sodium phosphate, 400 mM NaCl, 100 mM KCI, 0.5% Triton-X, pH 7.8) containing Complete EDTA Protease Inhibitor Cocktail tablets (Roche) at 1 tablet/50 ml buffer. Lysis buffer was used at 5 ml buffer per gram of bacterial pellet. Using a homogenizer, bacterial cells were homogenized at 4-20 sec intervals with 30 sec on ice between each interval. Lysozyme/DNasc/RNase cocktail was added at 50 ul/10 ml lysis buffer. The homogenized cells were then incubated on ice for 30 min. Lysate was cleared by centrifugation at 10,000 rpm for 20 min at 4° C. and filtered using a 0.45 um PES filter. Lysate was buffer exchanged with Equilibration Buffer (20 mM sodium phosphate, 0. M NaCl, pH 7.4) using a 10,000 mwco centrifugal filter (Millipore).

EF-Tu was purified using a 1 ml His-Trap excel column (Cytiva). The column was equilibrated with 5 CV (column volumes) of Equilibration Buffer and once the protein was loaded on to the column, the column was washed with 20 CV of Wash Buffer (20 mM sodium phosphate, 0.5 M NaCl, 10 mM imidazole, pH 7.4). The protein was eluted with 5 CV of Elution Buffer (20 mM sodium phosphate, 0.5 M NaCl, 500 mM imidazole, pH 7.4) and buffer exchanged with 1× PBS using a 10,000 mwco centrifugal filter (Millipore).Ten micrograms of protein was analyzed by SDS-Page on a 4-20% tris/glycine gel (BioRad) and stained with Coomassic Blue. One microgram of protein was used for his-tag detection by Western blotting using the iBLOT system (Invitrogen). A 1:2000 dilution of the primary antibody 6×His Tag mouse IgG2b (Thermo Scientific), and 1:2500 of the secondary antibody anti-mouse HRP (Promega) were used for his-tag binding. Detection was with Clarity Western ECL Substrate (BioRad). Size exclusion chromatography using FPLC (GE) was performed on the his-tag purified EF-Tu. The Enterokinase Cleavage Capture kit (Novagen) was used to remove the his-EK tag from the N-terminus of the EF-Tu protein. Recombinant enterokinase at a concentration of 1U/ul was used in a reaction mixture to cleave the his-EK tag from 750 μg of protein in a total volume of 750 ul. The reaction was incubated at 22° C. for 16 h. To determine completion of his-tag cleavage, 10 ug of protein was analyzed by SDS-Page and Western blot as described previously.

### Antigen Expression

BG505 DS-SOSIP was expressed as previously described (151). E. coli (Taxonomy ID: 199310) HMA protein was expressed by GenScript. Recombinant E. coli Ferric acrobactin receptor (iutA) was purchased from MyBioSource (catalog #MBS1137774). The gene encoding a rationally designed MenX construct with a 6×HisTag was synthesized by Genscript and cloned in the pET9a bacterial expression vector. For protein purification, MenX was purified following the same protocol as for EF-Tu (details in section above). One exception was that 50 μg/mL of Kanamycin was used in place of ampicillin. Sars2-HP and Sars2-2P plasmids were transiently transfected in Expi293F cells using polyethylenimine or ExpiFectamineTM transfection reagent (Thermo Fisher Scientific) and encoded the following: residues 1-1208 of the SARS-COV-2 spike with a mutated S1/S2 cleavage site, proline substitutions at positions 817, 892, 899, 942, 986 and 987, and a C-terminal T4-fibritin trimerization motif, an 8× HisTag, and a TwinStrepTag (SARS-CoV-2 S Hexapro (HP)); residues 1-1190 of the SARS-COV spike with proline substitutions at positions 968 and 969, and a C-terminal T4-fibritin trimerization motif, an 8× HisTag, and a TwinStrepTag (SARS-COV S-2P). All Expi293F cells were cultured at 8% CO2 saturation and 37° C. with shaking in FreeStyle F17 expression media (Thermo Fisher) supplemented to a final concentration of 0.1% Pluronic Acid F-68 and 4 mM L-glutamine. All coronavirus spike supernatants were collected 5-7 days post transfection, sterile filtered, and purified over a StrepTrap XT column (Cytiva Life Sciences). Purified proteins were further purified using a size exclusion Superose6 Increase column (Cytiva Life Sciences). The cctodomain of HCMV gB (Merlin strain), residues 1-698, with a 6-His tag at the C terminus was transiently expressed in Expi293 cells with PEI. His-tagged gB was purified by nickel affinity chromatography using an equilibrated, 1 mL pre-packed HisTrap HP column (GE Healthcare, IL, USA). The column was washed with 15mL of binding buffer, and purified protein was cluted from the column with binding buffer +0.5 M Imidazole, pH=7.4. His-tagged gB was further purified by gel filtration using a Superose 6 Increase 10/300 GL column, and eluted fractions corresponding to correctly folded protein were collected for further analysis. RSV-F plasmid was a gift from Jason Mclellan's lab. RSV Post Fusion was transiently expressed in Expi293 cells with Expifectamine 293 reagent. His-tagged RSV Post was purified by nickel affinity chromatography using an equilibrated, 1 mL pre-packed HisTrap HP column (GE Healthcare, IL, USA). The column was washed with 15 mL of binding buffer, and purified protein was eluted from the column with binding buffer +0.5 M Imidazole, pH=7.4. His-tagged gB was further purified by gel filtration using a Superose 6 Increase 10/300 GL column, and eluted fractions corresponding to correctly folded protein were collected for further analysis. VAR2CSA was a gift from Dr. Maria del Pilar Quintana. The protein was expressed in FreeStyle 293F cells and purified by his-tag affinity chromatography as previously described.

### Autoreactivity Assays

Monoclonal antibody autoreactivity was measured using the The ZEUS AtheNA Multi-Lyte ANA-II Plus Test System per manufacturer's instructions (ZEUS Scientific). Reactivity of each mAb was determined at multiple concentrations against eight separate autoantigens (SSA, SSB, Sm, RNP, Scl-70, Jo-1, Centromere B, and Histone). Anti-cardiolipin reactivity of the mAbs was measured at multiple concentrations using QUANTA Lite ACA IgG III (Werfen) per manufacturer's instructions.

### Animal Immunizations

Male and female, four-to eight-week-old, Hartley guinea pigs (GPs) (n=6/group) were used for these studies. All procedures were conducted according to protocols approved by Cocalico Biologics, INC. Animal Care and Use Committee. GPs were immunized day 0 (100 μg EF-Tu or 1×PBS) and received booster injections with BG505 DS-SOSIP 100 μg, days 28 and 56. All injection were intramuscular. Purified protein was prepared in 1×PBS. BG505 DS-SOSIP protein was emulsified 1:1 in TiterMax Gold (Sigma-Aldrich, MO, USA). Blood and stool were collected before each injection and at the conclusion, day 84. Serum and stool were stored at −80° C. for further analysis. Spleenocytes were collected at day 84 and stored at −80° C. for further analysis.

### Statistical Analysis

All statistical analyses and graphing were done using GraphPad Prism 9. Significance between pairwise combinations of immunization groups was determined by Mann-Whitney U test. ELISA EC50 values were determined by interpolating a standard curve using GraphPad Prism 9.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### TABLES

TABLE 1
HIV-1 mAb panel used to screen against commensal lysate.

| Antibody | Epitope | Source | Classification |
|---|---|---|---|
| VRC01 | CD4bs | Infection | bNAb |
| 17b | CD4i | Infection | Non- bNAb |
| PG9 | V1V2 | Infection | bNAb |
| CH58 | V1V2 | Vaccination | Non- bNAb |
| 830A | V1V2 | Infection | NAb |
| 2158 | V1V2 | Infection | NAb |
| PGT145 | V1V2 | Infection | bNAb |
| Z13e1 | MPER | Infection | NAb |
| 10E8 | MPER | Infection | bNAb |
| VRC34 | FP | Infection | bNAb |
| 3074 | V3 | Infection | NAb |
| PGT121 | V3 | Infection | bNAb |

TABLE 2
Computational analysis of mass spectrometry hits comparing CH58 epitope region to all
residues of target sequences.

| | | | SEQ | | SEQ |
|---|---|---|---|---|---|
| | Identity | | ID | Peptide from HIV-1 | ID |
| Sequence ID | (%) | Peptide from targets | NO. | strains | NO. |
| tr[A5VLH1]A5 | 64.3 | VEKTVEHFGDLDVI | 8 | VQKTYAFFYDLDVI | 14 |
| r-tr[A5VI67] | 57.1 | MGYELAQVYDLDVP | 9 | MKKEYALFYDLDVI | 15 |
| sp[A5VLK6]RS | 57.1 | TPKTVDSLMKLDLP | 10 | TKTVYSLFYKLDIV | 16 |
| tr[A5VHJ7]A5 | 57.1 | KQLALDMFGSKDDI | 11 | KQLAYALFYSLDII | 17 |
| tr[A5VIB7]A5 | 57.1 | EEKKQNAQKLVDDL | 12 | EEKKQALFYVSDLV | 18 |
| tr[A5VLH1]A5 | 57.1 | VEKTVEHFGDLDVI | 8 | VEKQNVLFYKDVI | 19 |
| tr[A5VLH1]A5 | 57.1 | VEKTVEHFGDLDVI | 8 | VEKNHAIFYDLDVM | 20 |
| r-tr[A5VI67] | 50.0 | PMGYELAQVYDLDV | 13 | MKKEYALFDLDVI | 21 |
| r-tr[A5VI67] | 50.0 | PMGYELAQVYDLDV | 13 | MQKETAQFYKLDIV | 22 |
| r-tr[A5VI67] | 50.0 | MGYELAQVYDLDVP | 9 | MQKEYALFYKLDVV | 23 |
| r-tr[A5VI67] | 50.0 | MGYELAQVYDLDVP | 9 | MQKEYAIFYNLDVV | 24 |
| r-tr[A5VI67] | 50.0 | MGYELAQVYDLDVP | 9 | MNREYALYDTLDVV | 25 |
| r-tr[A5VI67] | 50.0 | MGYELAQVYDLDVP | 9 | MQKEYAILYKLDVT | 26 |
| r-tr[A5VI67] | 50.0 | MGYELAQVYDLDVP | 9 | MKKEYATFYRLDVV | 27 |
| r-tr[A5VI67] | 50.0 | MGYELAQVYDLDVP | 9 | MQKEYAVFYKLDVV | 28 |
| r-tr[A5VI67] | 50.0 | MGYELAQVYDLDVP | 9 | MQKEYALFYKLDVQ | 29 |
| r-tr[A5VI67] | 50.0 | MGYELAQVYDLDVP | 9 | MQTEYALFYKLDVM | 30 |
| r-tr[A5VI67] | 50.0 | MGYELAQVYDLDVP | 9 | MRKEYALFYRLDVV | 31 |
| r-tr[A5VI67] | 50.0 | MGYELAQVYDLDVP | 9 | MHKEYALFYKLDVT | 32 |
| r-tr[A5VI67] | 50.0 | MGYELAQVYDLDVP | 9 | MQKEYALFYKLDVT | 33 |

TABLE 3
Computational analysis of mass spectrometry hits comparing CH58 epitope to epitope
residues of target sequences.

| | | | SEQ | | SEQ |
|---|---|---|---|---|---|
| | Identity | | ID | Peptide from HIV-1 | ID |
| Sequence ID | (%) | Peptide from targets | NO. | strains | NO. |
| sp[A5VL03]SY | 85.7 | LEKVNYNLIRIDYL | 34 | KXKVNXXLYRXDXX | 37 |
| sp[A5VLG3]GA | 85.7 | VPKEYFDGLDEDVQ | 35 | VXKEYXXFYDXDXX | 38 |
| tr[A5VHJ7]A5 | 85.7 | KQLALDMFGSKDDI | 11 | KXLAYXXFYSXDXX | 39 |
| tr[A5VLH1]A5 | 85.7 | VEKTVEHFGDLDVI | 8 | VXKTYXXFYDXDXX | 40 |
| r-tr[A5VI67] | 78.6 | MGYELAQVYDLDVP | 9 | MXKEYXXFYDXDXX | 41 |
| r-tr[A5VI67] | 78.6 | KEKAQMEFREKQDI | 36 | KXKAYXXFYKXDXX | 42 |
| r-tr[A5VI67] | 78.6 | KEKAQMEFREKQDI | 36 | KXKANXXFYKXDXX | 43 |
| r-tr[A5VI67] | 78.6 | KEKAQMEFREKQDI | 36 | KXKVQXXFYKXDXX | 44 |
| r-tr[A5VI67] | 78.6 | KEKAQMEFREKQDI | 36 | KXKVQXXFYKXDXX | 44 |
| r-tr[A5VI67] | 78.6 | KEKAQMEFREKQDI | 36 | KXKAYXXFYKXDXX | 42 |
| r-tr[A5VI67] | 78.6 | KEKAQMEFREKQDI | 36 | KXKAYXXFYKXDXX | 42 |
| r-tr[A5VI67] | 78.6 | KEKAQMEFREKQDI | 36 | KXKAYXXFYKXDXX | 42 |
| r-tr[A5VI67] | 78.6 | KEKAQMEFREKQDI | 36 | KXKMQXXFYKXDXX | 45 |
| r-tr[A5VI67] | 78.6 | KEKAQMEFREKQDI | 36 | KXKAYXXFYKXDXX | 42 |
| r-tr[A5VI67] | 78.6 | KEKAQMEFREKQDI | 36 | KXKAYXXFYKXDXX | 42 |
| r-tr[A5VI67] | 78.6 | KEKAQMEFREKQDI | 36 | KXKVQXXFYKXDXX | 44 |
| r-tr[A5VI67] | 78.6 | KEKAQMEFREKQDI | 36 | KXKAYXXFYKXDXX | 42 |
| r-tr[A5VI67] | 78.6 | KEKAQMEFREKQDI | 36 | KXKAYXXFYKXDXX | 42 |
| r-tr[A5VI67] | 78.6 | KEKAQMEFREKQDI | 36 | KXKVQXXFYKXDXX | 44 |
| r-tr[A5VI67] | 78.6 | KEKAQMEFREKQDI | 36 | KXKAYXXFYKXDXX | 42 |

TABLE 4
EF-Tu peptides matched to CH58 Env epitope region.

| Peptide Name | Peptide Sequence | SEQ ID NO. |
|---|---|---|
| EF-Tu_1 | AKGLAKAEDYADID | 1 |
| EF-Tu_2 | KGLAKAEDYADIDA | 2 |
| EF-Tu_3 | VGVQYIVVFLNKTD | 3 |
| EF-Tu_4 | VQYIVVFLNKTDLV | 4 |
| EF-Tu_5 | TVTGLEMFHKTLDL | 5 |
| EF-Tu_6 | MTKEEGGRHTPFFS | 6 |
| EF-Tu_7 | ELRDKKQKVHALFKLDIV | 7 |

TABLE 5
Consensus CH58 epitopes

| CH58 Epitope | SEQ ID | Epitope resides | |
|---|---|---|---|
| Region | NO. | (underlined) | SEQ ID NO. |
| KKKVHALFYKLDIV | 46 | KXKVHXXFYKXDIX | 47 |

TABLE 6
Prioritized Mass Spectrometry Hits

| Protein | Molecular Weight | % Identity |
|---|---|---|
| Elongation Factor Tu | 43 kDa | 78 |
| ATP Synthase subunit β | 52 kDa | 78 |
| Na⁺ dependent nucleoside | 44 kDa | 78 |
| transporter | | |

TABLE 7
HIV Envelope Sequences

| HIV | | |
|---|---|---|
| Envelope | | |
| (Env) | | |
| Sequence | | |
| name | Sequence | SEQ ID NO. |
| BG505 | MPMGSLQPLATLYLLGMLVASVLAAENLWVTVYYGVPVWKDAET | 48 |
| T332N. | TLFCASDAKAYETEKHNVWATHACVPTDPNPQEIHLENVTEEFNMW | |
| DS. | KNNMVEQMHTDIISLWDQSLKPCVKLTPLCVTLQCTNVTNNITDDM | |
| SOSIP.sc. | RGELKNCSFNMTTELRDKKQKVYSLFYRLDVVQINENQGNRSNNSN | |
| 664 | KEYRLINCNTSACTQACPKVSFEPIPIHYCAPAGFAILKCKDKKFNGT | |
| | GPCPSVSTVQCTHGIKPVVSTQLLLNGSLAEEEVMIRSENITNNAKNI | |
| | LVQFNTPVQINCTRPNNNTRKSIRIGPGQAFYATGDIIGDIRQAHCNVS | |
| | KATWNETLGKVVKQLRKHFGNNTIIRFANSSGGDLEVTTHSFNCGGE | |
| | FFYCNTSGLFNSTWISNTSVQGSNSTGSNDSITLPCRIKQIINMWQRIG | |
| | QCMYAPPIQGVIRCVSNITGLILTRDGGSTNSTTETFRPGGGDMRDN | |
| | WRSELYKYKVVKIEPLGVAPTRCKRRVVGGGSGGGGSGGGGSGGA | |
| | VGIGAVFLGFLGAAGSTMGAASMTLTVQARNLLSGIVQQQSNLLRA | |
| | PEAQQHLLKLTVWGIKQLQARVLAVERYLRDQQLLGIWGCSGKLIC | |
| | CTNVPWNSSWSNRNLSEIWDNMTWLQWDKEISNYTQIIYGLLEESQ | |
| | NQQEKNEQDLLALDGSGGSGSGGSGSGLNDIFEAQKIEWHE | |
| 286.36. | MPMGSLQPLATLYLLGMLVASVLAGEDLWVTVYYGVPVWKEANPT | 49 |
| DS. | LFCASDAKAYKTEMHNVWATHACVPTDPNPQEMVLENVTEDFNM | |
| SOSIP.sc. | WKNGMVEQMHQDIISLWDQSLKPCVKLTPLCVTLNCTEVTRSSNGTI | |
| 664 | NNNSTEMKNCSFNVTTDLRDKKKKEHALFYRLDIVPLDETNGTSSEY | |
| | RLINCNTSTCTQACPKVSFDPIPIHYCAPAGYAILKCKDKKFNGTGPC | |
| | KNVSTVQCTHGIKPVVSTQLLLNGSIAEGEIIIRSENLTNNAKIIIVQLN | |
| | VTVEINCTRPNNNTRRSIRIGPGQTFYATGEIIGDIRQAHCNISREKWN | |
| | RTLQKVEKKLEELFPNKTIHFTSSSGGDLEITTHSFNCMGEFFYCNTS | |
| | ALFNNNNDSTNSNITLPCRIRQFINMWQEVGRCMYAPPIQGVITCKSN | |
| | VTGLLLTRDGGIINDTEIFRPGGGDMRDNWRSELYKYKVVEIKPLGIA | |
| | PTTCKRRVVEGGSGGGGSGGGGSGGAVGIGAVFLGFLGAAGSTMGA | |
| | ASITLTAQARQLLSGIVQQQSNLLRAPEAQQHMLQLTVWGIKQLQTR | |
| | VLAIERYLKDQQLLGIWGCSGKLICCTAVPWNGSWSNKSQDEIWHN | |
| | MTWMQWDKEINNYTNIIYGLLEVSQNQQEKNEQDLLALDGSGGSGS | |
| | GGSGSGLNDIFEAQKIEWHE | |
| 5768.04. | MPMGSLQPLATLYLLGMLVASVLAADKLWVTVYYGVPVWKETTTT | 50 |
| DS.SOSIP. | LFCASDARAYDTEVHNVWATHACVPTDPNPQEVVLGNVTENFNMW | |
| sc. | KNNMVEQMHEDIISLWDQSLKPCVRLTPLCVTLNCIDYYGNTTNSN | |
| 664 | NSSETMMEKGEIKNCSFNITTRLKDKMQKEYALFYKYDIVPIDNRVG | |
| | NDTSNATSYRLTSCNTSVCTQACPKVSFEPIPIHYCAPAGFAILKCND | |
| | KKFNGTGPCKNVSTVQCTHGIKPVVSTQLLLNGSLAEEEVMIRSENF | |
| | TDNAKTIIVQLNETVEINCTRPNNNTRKSIHMGPGKVFYTTGEIIGDIR | |
| | QAHCNINRAKWNNTLIKIVEKLRVKFNKTISFKQSSGGDPEIEMHSFN | |
| | CGGEFFYCNTTQLFNSTWENNATLNVNSNVTEGSENITLPCRIRQIVN | |
| | MWQEVGKCMYAPPIQGQIRCSSNITGLLLTRDGGGSNSSNTSEEVFR | |
| | PGGGNMRDNWRSELYKYKVVKIEPLGIAPTKCKRRVVQGGSGGGGS | |
| | GGGGSGGTVGIGALFLGFLGAAGSTMGAASMTLTVQARQLLSGIVQ | |
| | QQNNLLRAPQAQQHLLQLTVWGIKQLQARVLAVERYLKDQQLLGI | |
| | WGCSGKLICCTAVPWNASWSNKSLNEIWDNMTWMEWEKEIDNYTS | |
| | LIYTLIEESQNQQEKNEQELLELDGSGGSGSGGSGSGLNDIFEAQKIE | |
| | WHE | |
| CZA97. | MPMGSLQPLATLYLLGMLVASVLAVGNMWVTVYYGVPVWTDAKT | 51 |
| DS.SOSIP. | TLFCASDTKAYDREVHNVWATHACVPTDPNPQEIVLENVTENFNM | |
| sc. | WKNDMVDQMHEDIISLWDQSLKPCVKLTPLCVTLHCTNATFKNNVT | |
| 664 | NDMNKEIRNCSFNTTTEIRDKKQQGYALFYRPDIVLLKENRNNSNNS | |
| | EYILINCNASTCTQACPKVNFDPIPIHYCAPAGYAILKCNNKTFSGKGP | |
| | CNNVSTVQCTHGIKPVVSTQLLLNGSLAEKEIIIRSENLTDNVKTIIVH | |
| | LNKSVEIVCTRPNNNTRKSMRIGPGQTFYATGDIIGDIRQAYCNISGS | |
| | KWNETLKRVKEKLQENYNNNKTIKFAPSSGGDLEITTHSFNCRGEFF | |
| | YCNTTRLFNNNATEDETITLPCRIKQIINMWQGVGRCMYAPPIAGNIT | |
| | CKSNITGLLLVRDGGEDNKTEEIFRPGGGNMKDNWRSELYKYKVIEL | |
| | KPLGIAPTGCKRRVVEGGSGGGGSGGGGSGGAVGIGAVFLGFLGAA | |
| | GSTMGAASLTLTVQARQLLSSIVQQQSNLLRAPEAQQHMLQLTVWG | |
| | IKQLQTRVLAIERYLKDQQLLGIWGCSGKLICCTNVPWNSSWSNKSQ | |
| | TDIWNNMTWMEWDREISNYTDTIYRLLEDSQTQQEKNEKDLLALDG | |
| | SGGSGSGGSGSGLNDIFEAQKIEWHE | |
| DU172. | MKAKLLVLLCTFTATYAGNLWVTVYYGVPVWKEAKTTLFCASDAK | 52 |
| 17.DS. | AHKEEVHNIWATHACVPTDPNPQEIVLKNVTENFNMWKNDMVDQ | |
| SOSIP. | MHEDIISLWDQSLKPCVKLTPLCVTLNCSDVKIKGTNATYNNATYNN | |
| sc.664 | NNTISDMKNCSFNTTTEITDKKKKEYALFYKLDVVALDGKETNSTNS | |
| | SEYRLINCNTSACTQACPKVSFDPIPIHYCAPAGYAILKCNNKTFNGT | |
| | GPCNNVSTVQCTHGIKPVVSTQLLLNGSLAEEEVVIRFENLTNNAKIII | |
| | VHLNESVEINCTRPSNNTRKSVRIGPGQTFFATGDIIGDIRQAHCNISR | |
| | KKWNTTLQRVKEKLKEKFPNKTIQFAPSSGGDLEITTHSFNCRGEFFY | |
| | CYTSDLFNSTYMSNNTGGANITLQCRIKQIIRMWQGVGQCMYAPPIA | |
| | GNITCKSNITGLLLTRDGGKEKNDTETFRPGGGDMRDNWRSELYKY | |
| | KVVEIKPLGIAPDKCKRRVVEGGSGGGGSGGGGSGGAVGIGAVFLGF | |
| | LGAAGSTMGAASMTLTVQARQLLSGIVQQQSNLLRAPEAQQHMLQ | |
| | LTVWGIKQLQTRVLAIERYLKDQQLLGIWGCSGKLICCTAVPWNAS | |
| | WSNKSYEEIWGNMTWMQWDREINNYTNTIYSLLEESQNQQEKNEK | |
| | DLLALDGSGGSGSGGSGSGLNDIFEAQKIEWHE | |
| HT593. | MPMGSLQPLATLYLLGMLVASVLATEKLWVTVYYGVPVWKEATTT | 53 |
| 1.DS. | LFCASDAKAYETEVHNVWATHACVPTDPNPQEVLLENVTENFNMW | |
| SOSIP. | KNNMVEQMQEDIISLWDQSLKPCVKLTPLCVTLECHDVNVNGTANN | |
| sc.664 | GTTNVTESGVNSSDVTSNNVTNSNWGTMEKGEIKNCSFNITTNIRDK | |
| | MQKETAQFYKLDIVPIEDQNKTNNTLYRLINCNTSVCTQACPKVSFE | |
| | PIPIHYCTPAGFAILKCNDRNFNGTGPCKNVSTVQCTHGIKPVVSTQL | |
| | LLNGSLAEAEVVIRSENFINNAKTIIIQLNETVEINCTRPNNNTSKRISI | |
| | GPGRAFRATKIIGNIRQAHCNISRATWNSTLKKIVAKLREQFGNKTIV | |
| | FQPSSGGDPEIVMHSFNCGGEFFYCNTTQLFNSTWNSTEESNSTEEGT | |
| | ITLPCRIKQIINMWQEVGKCMYAPPIEGQIRCSSNITGLLLTRDGGNN | |
| | NKTNGTEIFRPGGGDMRDNWRSELYKYKVVKIEPLGVAPTKCKRRV | |
| | VQGGSGGGGSGGGGSGGAVGIVGAMFLGFLGAAGSTMGAASMTLT | |
| | VQARLLLSGIVQQQNNLLRAPEAQQHLLQLTVWGIKQLQARVLAVE | |
| | RYLKDQQLLGIWGCSGKLICCTTVPWNTSWSNKSLSEIWDNMTWM | |
| | QWEREIDNYTSLIYTLIEESQNQQEKNEQELLELDGSGGSGSGGSGSG | |
| | LNDIFEAQKIEWHE | |
| KNH1209. | MPMGSLQPLATLYLLGMLVASVLATDNLWVTVYYGVPVWKDAET | 54 |
| 18. | TLFCASDAKAYATEKHNVWATHACVPTDPNPQEIHLENVTEEFNMW | |
| DS.SOSIP. | KNNMVEQMHTDIISLWDQSLKPCVKLTPLCVTLSCSNAKVSYSNAT | |
| sc. | VNNTIQDEIKNCSFNTTTVLRDKRQKVYSLFYRLDIVQIDNSSSDSSSS | |
| 664 | EYRLINCNTSACTQACPKVTFEPIPIHYCAPAGFAILKCKDEEFNGTGP | |
| | CKNVSTVQCTHGIKPVVSTQLLLNGSLAKREVKIRSENITNNAKNIIV | |
| | QFVDPVEINCTRPNNNTRKSIHIGPGQAFYATGDIIGDIRQAHCNVSRS | |
| | SWNKTLQQVAKQLGTYFKNKTIVFNTSSGGDPEITTHSFNCAGEFFY | |
| | CDTSGLFNSSWNDTTWKESNSTGSNDTITLLCRIKQIINMWQRTGQC | |
| | MYAPPIPGLISCKSNITGIILTRDGGNSHRTEETFRPGGGDMRDNWRS | |
| | ELYRYKVVQIEPLGVAPTRCRRRVVQGGSGGGGSGGGGSGGAVGIG | |
| | AVFLGFLGAAGSTMGAASITLTVQARQLLSGIVQQQSNLLRAPEAQQ | |
| | HLLKLTVWGIKQLQARVLAVERYLRDQQLLGIWGCSGKLICCTNVP | |
| | WNSSWSNKSYNDIWDNMTWLQWDKEIHNYTQLIYNLIEESQNQQE | |
| | KNEQDLLALDGSGGSGSGGSGSGLNDIFEAQKIEWHE | |
| MB539. | MPMGSLQPLATLYLLGMLVASVLAAENLWVTVYYGVPVWRDADT | 55 |
| 2B7.DS. | TLFCASDAKAYETEKHNVWATHACVPTDPNPQEIDLKNVTEEFNMW | |
| SOSIP. | KNNMVEQMHTDIISLWDQSLKPCVKLTPLCVTLNCSNANVTSENSTI | |
| sc.664 | MGDREEIKNCSFNMTTELRDKRQKVYSLFYRLDVVQINENQGNSSN | |
| | NNYSEYRLINCNTSACTQACPKVSFEPIPIHYCAPAGFAILKCKDEEFN | |
| | GTGPCKNVSTVQCTHGIKPVVSTQLLLNGSTAEKEIKIRSENITNNAKI | |
| | IIVQLVKPVIINCTRPNNNTRRSVHIGPGQAFYATGDIIGNIRQAYCTV | |
| | NRTDWNNTLQQVAKQLGKHFENKTIIFTKSSGGDLEITTHSFNCGGE | |
| | FFYCNTSSLFNSTWSHNNSTLLGSNSTESNETITLPCRIKQIVNMWQR | |
| | TGQCMYAPPIKGVIMCVSNITGLILTRDGGNDNSTNENETFRPGGGD | |
| | MRDNWRSELYKYKVVQIEPLGVAPTRCKRRVVEGGSGGGGSGGGG | |
| | SGGAVGIGAVFLGFLGAAGSTMGAASITLTVQARQLLSGIVRQQSNL | |
| | LRAPEAQQHLLKLTVWGIKQLQARVLAVERYLRDQQLLGIWGCSGK | |
| | LICCTSVPWNSSWSNKSLDEIWENMTWLQWEKEINNYTGLIYSLLEE | |
| | SQNQQEKNEQDLLALDGSGGSGSGGSGSGLNDIFEAQKIEWHE | |
| BG505 | MPMGSLQPLATLYLLGMLVASVLAAENLWVTVYYGVPVWKDAET | 56 |
| K169E | TLFCASDAKAYETEKHNVWATHACVPTDPNPQEIHLENVTEEFNMW | |
| SOSIP. | KNNMVEQMHTDIISLWDQSLKPCVKLTPLCVTLQCTNVTNNITDDM | |
| 664.sc | RGELKNCSFNMTTELRDKEQKVYSLFYRLDVVQINENQGNRSNNSN | |
| | KEYRLINCNTSAITQACPKVSFEPIPIHYCAPAGFAILKCKDKKFNGTG | |
| | PCPSVSTVQCTHGIKPVVSTQLLLNGSLAEEEVMIRSENITNNAKNIL | |
| | VQFNTPVQINCTRPNNNTRKSIRIGPGQAFYATGDIIGDIRQAHCNVS | |
| | KATWNETLGKVVKQLRKHFGNNTIIRFANSSGGDLEVTTHSFNCGGE | |
| | FFYCNTSGLFNSTWISNTSVQGSNSTGSNDSITLPCRIKQIINMWQRIG | |
| | QAMYAPPIQGVIRCVSNITGLILTRDGGSTNSTTETFRPGGGDMRDN | |
| | WRSELYKYKVVKIEPLGVAPTRCKRRVVGGGSGGGGSGGGGSGGA | |
| | VGIGAVFLGFLGAAGSTMGAASMTLTVQARNLLSGIVQQQSNLLRA | |
| | PEAQQHLLKLTVWGIKQLQARVLAVERYLRDQQLLGIWGCSGKLIC | |
| | CTNVPWNSSWSNRNLSEIWDNMTWLQWDKEISNYTQIIYGLLEESQ | |
| | NQQEKNEQDLLALDGSGGSGSGGSGSGLNDIFEAQKIEWHE | |
| BG505 | MPMGSLQPLATLYLLGMLVASVLAAENLWVTVYYGVPVWKDAET | 57 |
| D368R + | TLFCASDAKAYETEKHNVWATHACVPTDPNPQEIHLENVTEEFNMW | |
| N279K | KNNMVEQMHTDIISLWDQSLKPCVKLTPLCVTLQCTNVTNNITDDM | |
| SOSIP. | RGELKNCSFNMTTELRDKKQKVYSLFYRLDVVQINENQGNRSNNSN | |
| 664.sc | KEYRLINCNTSAITQACPKVSFEPIPIHYCAPAGFAILKCKDKKFNGTG | |
| (Double | PCPSVSTVQCTHGIKPVVSTQLLLNGSLAEEEVMIRSENITKNAKNIL | |
| knock- | VQFNTPVQINCTRPNNNTRKSIRIGPGQAFYATGDIIGDIRQAHCNVS | |
| out | KATWNETLGKVVKQLRKHFGNNTIIRFANSSGGRLEVTTHSFNCGGE | |
| (DKO)) | FFYCNTSGLFNSTWISNTSVQGSNSTGSNDSITLPCRIKQIINMWQRIG | |
| | QAMYAPPIQGVIRCVSNITGLILTRDGGSTNSTTETFRPGGGDMRDN | |
| | WRSELYKYKVVKIEPLGVAPTRCKRRVVGGGSGGGGSGGGGSGGA | |
| | VGIGAVFLGFLGAAGSTMGAASMTLTVQARNLLSGIVQQQSNLLRA | |
| | PEAQQHLLKLTVWGIKQLQARVLAVERYLRDQQLLGIWGCSGKLIC | |
| | CTNVPWNSSWSNRNLSEIWDNMTWLQWDKEISNYTQIIYGLLEESQ | |
| | NQQEKNEQDLLALDGSGGSGSGGSGSGLNDIFEAQKIEWHE | |
| BG505 | MPMGSLQPLATLYLLGMLVASVLAAENLWVTVYYGVPVWKDAET | 58 |
| N332T | TLFCASDAKAYETEKHNVWATHACVPTDPNPQEIHLENVTEEFNMW | |
| SOSIP. | KNNMVEQMHTDIISLWDQSLKPCVKLTPLCVTLQCTNVTNNITDDM | |
| 664.sc | RGELKNCSFNMTTELRDKKQKVYSLFYRLDVVQINENQGNRSNNSN | |
| | KEYRLINCNTSAITQACPKVSFEPIPIHYCAPAGFAILKCKDKKFNGTG | |
| | PCPSVSTVQCTHGIKPVVSTQLLLNGSLAEEEVMIRSENITNNAKNIL | |
| | VQFNTPVQINCTRPNNNTRKSIRIGPGQAFYATGDIIGDIRQAHCTVSK | |
| | ATWNETLGKVVKQLRKHFGNNTIIRFANSSGGDLEVTTHSFNCGGEF | |
| | FYCNTSGLFNSTWISNTSVQGSNSTGSNDSITLPCRIKQIINMWQRIGQ | |
| | AMYAPPIQGVIRCVSNITGLILTRDGGSTNSTTETFRPGGGDMRDNW | |
| | RSELYKYKVVKIEPLGVAPTRCKRRVVGGGSGGGGSGGGGSGGAVG | |
| | IGAVFLGFLGAAGSTMGAASMTLTVQARNLLSGIVQQQSNLLRAPEA | |
| | QQHLLKLTVWGIKQLQARVLAVERYLRDQQLLGIWGCSGKLICCTN | |
| | VPWNSSWSNRNLSEIWDNMTWLQWDKEISNYTQIIYGLLEESQNQQ | |
| | EKNEQDLLALDGSGGSGSGGSGSGLNDIFEAQKIEWHE | |

TABLE 8
EF-Tu Sequences

| | Sequence | SEQ ID NO |
|---|---|---|
| EF-Tu Amino Acid | MAEKEHYERTKPHVNIGTIGHVDHGKTTLTAAITKVLAAKGLA | 59 |
| Sequence | KAEDYADIDAAPEEKERGITINTAHVEYETEKRHYAHIDAPGH | |
| | ADYVKNMITGAAQMDGAILVVAATDGPMPQTREHILLARQVG | |
| | VQYIVVFLNKTDLVDDDELVDLVEMEVRDLLSEYDFPGDDVP | |
| | VVRGSALKALEGDPEQEKVILHLMDVIDDYIPTPKRPTDKPFM | |
| | MPVEDVFTITGRGTVASGRIDRGTVKVGDEVEIVGLTEDVLKST | |
| | VTGLEMFHKTLDLGEAGDNVGVLLRGISHDQIQRGQVLAEPGS | |
| | IQTHKNFKGEVYVMTKEEGGRHTPFFSNYRPQFYFHTTDVTGTI | |
| | ELPDGVEMVMPGDNVTFTVNLQKPVALEKGLKFTIREGGHTV | |
| | GAGVVSDILD | |
| EF-Tu DNA | ATGGCTGAAAAAGAACATTATGAACGTACAAAACCCCACGT | 60 |
| Sequence | TAACATTGGTACTATTGGCCACGTTGACCACGGGAAGACTA | |
| | CTTTAACTGCTGCTATTACAAAGGTATTGGCAGCCAAAGGTT | |
| | TAGCAAAGGCAGAAGATTACGCTGATATCGATGCTGCTCCA | |
| | GAAGAAAAGGAACGTGGTATCACTATCAACACTGCCCACGT | |
| | TGAATACGAAACTGAAAAGCGTCACTACGCACACATCGATG | |
| | CCCCTGGACACGCTGACTACGTTAAGAACATGATCACTGGT | |
| | GCTGCACAAATGGATGGTGCTATCCTTGTTGTTGCTGCTACT | |
| | GATGGTCCTATGCCACAAACTCGTGAACACATTCTTCTTGCT | |
| | CGTCAGGTTGGTGTTCAATACATCGTTGTATTCTTGAACAAG | |
| | ACTGACTTAGTTGACGATGATGAATTAGTTGACTTAGTTGAA | |
| | ATGGAAGTTCGTGACTTACTTAGCGAATACGATTTCCCTGGT | |
| | GACGATGTTCCAGTTGTTCGTGGATCTGCTCTTAAGGCGCTT | |
| | GAAGGTGACCCAGAACAAGAAAAAGTTATCCTTCACTTAAT | |
| | GGATGTTATTGATGACTACATCCCAACTCCAAAGCGTCCTAC | |
| | TGACAAGCCATTCATGATGCCTGTCGAAGACGTATTTACTAT | |
| | CACTGGTCGTGGTACTGTTGCTTCTGGTCGTATTGACCGTGG | |
| | TACTGTTAAGGTCGGTGACGAAGTTGAAATCGTTGGTTTAAC | |
| | TGAAGACGTTCTTAAGTCAACTGTTACTGGTTTGGAAATGTT | |
| | CCACAAGACTCTTGATCTTGGTGAAGCCGGTGATAACGTTGG | |
| | TGTACTTCTTCGTGGTATTTCACACGACCAAATCCAACGTGG | |
| | TCAAGTTTTGGCTGAACCAGGTTCAATCCAAACTCACAAGA | |
| | ACTTCAAGGGTGAAGTTTACGTTATGACTAAGGAAGAAGGG | |
| | GGACGTCACACTCCATTCTTCTCAAACTACCGTCCACAATTC | |
| | TACTTCCACACAACTGATGTTACTGGTACTATCGAATTACCA | |
| | GATGGTGTAGAAATGGTTATGCCTGGTGATAACGTTACATTC | |
| | ACTGTTAACTTACAAAAGCCAGTTGCTCTTGAAAAGGGTCTT | |
| | AAGTTCACTATTCGTGAAGGTGGACACACTGTTGGTGCCGGT | |
| | GTTGTATCCgCCATCTTAGACTAA | |

What is claimed is:

1. A method of preventing an infection from a human immunodeficiency virus (HIV), the method comprising administering to a subject an effective amount of a first composition comprising a commensal microbe antigen and an effective amount of a second composition comprising an HIV antigen.

2. The method of claim 1, wherein the first composition is administered before the second composition.

3. The method of claim 1, wherein the first composition is simultaneously administered with the second composition.

4. The method of claim 1, wherein the commensal microbe antigen is derived from an intestinal microbe.

5. The method of claim 4, wherein the intestinal microbe comprises Limosilactobacilius reuteri (L. reuteri) or Bacteroides thetaiotaomicron (B. thetaiotaomicron).

6. The method of claim 1, wherein the commensal microbe antigen comprises at least 80% sequence identity to SEQ ID NO: 7.

7. The method of claim 1, wherein the commensal microbe antigen comprises at least 90% sequence identity to SEQ ID NO: 7.

8. The method of claim 1, wherein the commensal microbe antigen comprises SEQ ID NO: 7.

9. The method of claim 1, wherein the commensal microbe antigen comprises Elongation Factor Tu (EF-Tu).

10. The method of claim 9, wherein EF-Tu comprises SEQ ID NO: 59, or a fragment thereof.

11. The method of claim 1, wherein the HIV antigen is derived from an HIV-1 virus.

12. The method of claim 1, wherein the HIV antigen comprises an HIV glycoprotein, or a fragment thereof.

13. The method of claim 1, wherein the HIV antigen comprises SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, or a fragment thereof.

14. The method of claim 12, wherein the HIV glycoprotein comprises a whole HIV envelope (Env) protein.

15. The method of claim 12, wherein the fragment of the HIV glycoprotein comprises a CD4 binding (CD4bs), a CD4 induced site (CD4i), a V1V2 loop region (V1V2), a variable region 3 (V3), a membrane proximal external region (MPER), or a fusion peptide (FP).

16. The method of claim 1, further comprising administering an adjuvant.

17. The method of claim 16, wherein the adjuvant comprises a water-in-oil adjuvant.

18. The method of claim 1, wherein the commensal microbe antigen and the HIV antigen enhances an immune response against the HIV relative to an HIV antigen alone.

19. The method of claim 18, wherein the immune response comprises an increased HIV antibody response relative to a control.

20. A method of boosting an immune response against a human immunodeficiency virus (HIV), the method comprising administering to a subject an effective amount of a first composition comprising a commensal microbe antigen and an effective amount of a second composition comprising an HIV antigen.


SEQUENCE LISTING (60 sequences)

SEQ ID NO: 1 - protein, 14 aa, Limosilactobacillus reuteri
  REGION 1..14: note = Description of sequence: EF-Tu_1
AKGLAKAEDYADID

SEQ ID NO: 2 - protein, 14 aa, Limosilactobacilius reuteri
  REGION 1..14: note = Description of sequence: EF-Tu_2
KGLAKAEDYADIDA

SEQ ID NO: 3 - protein, 14 aa, Limosilactobacilius reuteri
  REGION 1..14: note = Description of sequence: EF-Tu_3
VGVQYIVVFLNKTD

SEQ ID NO: 4 - protein, 14 aa, Limosilactobacilius reuteri
  REGION 1..14: note = Description of sequence: EF-Tu_4
VQYIVVFLNKTDLV

SEQ ID NO: 5 - protein, 14 aa, Limosilactobacilius reuteri
  REGION 1..14: note = Description of sequence: EF-Tu_5
TVTGLEMFHKTLDL

SEQ ID NO: 6 - protein, 14 aa, Limosilactobacilius reuteri
  REGION 1..14: note = Description of sequence: EF-Tu_6
MTKEEGGRHTPFFS

SEQ ID NO: 7 - protein, 18 aa, Limosilactobacilius reuteri
  REGION 1..18: note = Description of sequence: EF-Tu_7
ELRDKKQKVHALFKLDIV

SEQ ID NO: 8 - protein, 14 aa, synthetic construct
  REGION 1..14: note = Description of sequence: tr[A5VLH1]A5; peptide from target sequence
VEKTVEHFGDLDVI

SEQ ID NO: 9 - protein, 14 aa, synthetic construct
  REGION 1..14: note = Description of sequence: r-tr[A5VI67]; peptide from target sequence
MGYELAQVYDLDVP

SEQ ID NO: 10 - protein, 14 aa, synthetic construct
  REGION 1..14: note = Description of sequence: sp[A5VLK6]RS; peptide from target sequence
TPKTVDSLMKLDLP

SEQ ID NO: 11 - protein, 14 aa, synthetic construct
  REGION 1..14: note = Description of sequence: tr[A5VHJ7]A5; peptide from target sequence
KQLALDMFGSKDDI

SEQ ID NO: 12 - protein, 14 aa, synthetic construct
  REGION 1..14: note = Description of sequence: tr[A5VIB7]A5; peptide from target sequence
EEKKQNAQKLVDDL

SEQ ID NO: 13 - protein, 14 aa, synthetic construct
  REGION 1..14: note = Description of sequence: r-tr[A5VI67]; peptide from target sequence
PMGYELAQVYDLDV

SEQ ID NO: 14 - protein, 14 aa, synthetic construct
  REGION 1..14: note = Description of sequence: tr[A5VLH1]A5; peptide from HIV-1 strains
VQKTYAFFYDLDVI

SEQ ID NO: 15 - protein, 14 aa, synthetic construct
  REGION 1..14: note = Description of sequence: r-tr[A5VI67]; peptide from HIV-1 strains
MKKEYALFYDLDVI

SEQ ID NO: 16 - protein, 14 aa, synthetic construct
  REGION 1..14: note = Description of sequence: sp[A5VLK6]RS; peptide from HIV-1 strains
TKTVYSLFYKLDIV

SEQ ID NO: 17 - protein, 14 aa, synthetic construct
  REGION 1..14: note = Description of sequence: tr[A5VHJ7]A5; peptide from HIV-1 strains
KQLAYALFYSLDII

SEQ ID NO: 18 - protein, 14 aa, synthetic construct
  REGION 1..14: note = Description of sequence: tr[A5VIB7]A5; peptide from HIV-1 strains
EEKKQALFYVSDLV

SEQ ID NO: 19 - protein, 13 aa, synthetic construct
  REGION 1..13: note = Description of sequence: tr[A5VLH1]A5; peptide from HIV-1 strains
VEKQNVLFYKDVI

SEQ ID NO: 20 - protein, 14 aa, synthetic construct
  REGION 1..14: note = Description of sequence: tr[A5VLH1]A5; peptide from HIV-1 strains
VEKNHAIFYDLDVM

SEQ ID NO: 21 - protein, 13 aa, synthetic construct
  REGION 1..13: note = Description of sequence: r-tr[A5VI67]; peptide from HIV-1 strains
MKKEYALFDLDVI

SEQ ID NO: 22 - protein, 14 aa, synthetic construct
  REGION 1..14: note = Description of sequence: r-tr[A5VI67]; peptide from HIV-1 strains
MQKETAQFYKLDIV

SEQ ID NO: 23 - protein, 14 aa, synthetic construct
  REGION 1..14: note = Description of sequence: r-tr[A5VI67]; peptide from HIV-1 strains
MQKEYALFYKLDVV

SEQ ID NO: 24 - protein, 14 aa, synthetic construct
  REGION 1..14: note = Description of sequence: r-tr[A5VI67]; peptide from HIV-1 strains
MQKEYAIFYNLDVV

SEQ ID NO: 25 - protein, 14 aa, synthetic construct
  REGION 1..14: note = Description of sequence: r-tr[A5VI67]; peptide from HIV-1 strains
MNREYALYDTLDVV

SEQ ID NO: 26 - protein, 14 aa, synthetic construct
  REGION 1..14: note = Description of sequence: r-tr[A5VI67]; peptide from HIV-1 strains
MQKEYAILYKLDVT

SEQ ID NO: 27 - protein, 14 aa, synthetic construct
  REGION 1..14: note = Description of sequence: r-tr[A5VI67]; peptide from HIV-1 strains
MKKEYATFYRLDVV

SEQ ID NO: 28 - protein, 14 aa, synthetic construct
  REGION 1..14: note = Description of sequence: r-tr[A5VI67]; peptide from HIV-1 strains
MQKEYAVFYKLDVV

SEQ ID NO: 29 - protein, 14 aa, synthetic construct
  REGION 1..14: note = Description of sequence: r-tr[A5VI67]; peptide from HIV-1 strains
MQKEYALFYKLDVQ

SEQ ID NO: 30 - protein, 14 aa, synthetic construct
  REGION 1..14: note = Description of sequence: r-tr[A5VI67]; peptide from HIV-1 strains
MQTEYALFYKLDVM

SEQ ID NO: 31 - protein, 14 aa, synthetic construct
  REGION 1..14: note = Description of sequence: r-tr[A5VI67]; peptide from HIV-1 strains
MRKEYALFYRLDVV

SEQ ID NO: 32 - protein, 14 aa, synthetic construct
  REGION 1..14: note = Description of sequence: r-tr[A5VI67]; peptide from HIV-1 strains
MHKEYALFYKLDVT

SEQ ID NO: 33 - protein, 14 aa, synthetic construct
  REGION 1..14: note = Description of sequence: r-tr[A5VI67]; peptide from HIV-1 strains
MQKEYALFYKLDVT

SEQ ID NO: 34 - protein, 14 aa, synthetic construct
  REGION 1..14: note = Description of sequence: sp[A5VL03]SY; peptide from target sequence
LEKVNYNLIRIDYL

SEQ ID NO: 35 - protein, 14 aa, synthetic construct
  REGION 1..14: note = Description of sequence: sp[A5VLG3]GA; peptide from target sequence
VPKEYFDGLDEDVQ

SEQ ID NO: 36 - protein, 14 aa, synthetic construct
  REGION 1..14: note = Description of sequence: r-tr[A5VI67]; peptide from target sequence
KEKAQMEFREKQDI

SEQ ID NO: 37 - protein, 14 aa, synthetic construct
  REGION 1..14: note = Description of sequence: sp[A5VL03]SY
  SITE 2: note = X refers to a non-epitope amino acid residue
  SITE 6: note = X refers to a non-epitope amino acid residue
  SITE 7: note = X refers to a non-epitope amino acid residue
  SITE 11: note = X refers to a non-epitope amino acid residue
  SITE 13: note = X refers to a non-epitope amino acid residue
  SITE 14: note = X refers to a non-epitope amino acid residue
KXKVNXXLYRXDXX

SEQ ID NO: 38 - protein, 14 aa, synthetic construct
  REGION 1..14: note = Description of sequence: sp[A5VLG3]GA
  SITE 2: note = X refers to a non-epitope amino acid residue
  SITE 6: note = X refers to a non-epitope amino acid residue
  SITE 7: note = X refers to a non-epitope amino acid residue
  SITE 11: note = X refers to a non-epitope amino acid residue
  SITE 13: note = X refers to a non-epitope amino acid residue
  SITE 14: note = X refers to a non-epitope amino acid residue
VXKEYXXFYDXDXX

SEQ ID NO: 39 - protein, 14 aa, synthetic construct
  REGION 1..14: note = Description of sequence: tr[A5VHJ7]A5
  SITE 2: note = X refers to a non-epitope amino acid residue
  SITE 6: note = X refers to a non-epitope amino acid residue
  SITE 7: note = X refers to a non-epitope amino acid residue
  SITE 11: note = X refers to a non-epitope amino acid residue
  SITE 13: note = X refers to a non-epitope amino acid residue
  SITE 14: note = X refers to a non-epitope amino acid residue
KXLAYXXFYSXDXX

SEQ ID NO: 40 - protein, 14 aa, synthetic construct
  REGION 1..14: note = Description of sequence: tr[A5VHJ7]A5
  SITE 2: note = X refers to a non-epitope amino acid residue
  SITE 6: note = X refers to a non-epitope amino acid residue
  SITE 7: note = X refers to a non-epitope amino acid residue
  SITE 11: note = X refers to a non-epitope amino acid residue
  SITE 13: note = X refers to a non-epitope amino acid residue
  SITE 14: note = X refers to a non-epitope amino acid residue
VXKTYXXFYDXDXX

SEQ ID NO: 41 - protein, 14 aa, synthetic construct
  REGION 1..14: note = Description of sequence: r-tr[A5VI67]
  SITE 2: note = X refers to a non-epitope amino acid residue
  SITE 6: note = X refers to a non-epitope amino acid residue
  SITE 7: note = X refers to a non-epitope amino acid residue
  SITE 11: note = X refers to a non-epitope amino acid residue
  SITE 13: note = X refers to a non-epitope amino acid residue
  SITE 14: note = X refers to a non-epitope amino acid residue
MXKEYXXFYDXDXX

SEQ ID NO: 42 - protein, 14 aa, synthetic construct
  REGION 1..14: note = Description of sequence: r-tr[A5VI67]
  SITE 2: note = X refers to a non-epitope amino acid residue
  SITE 6: note = X refers to a non-epitope amino acid residue
  SITE 7: note = X refers to a non-epitope amino acid residue
  SITE 11: note = X refers to a non-epitope amino acid residue
  SITE 13: note = X refers to a non-epitope amino acid residue
  SITE 14: note = X refers to a non-epitope amino acid residue
KXKAYXXFYKXDXX

SEQ ID NO: 43 - protein, 14 aa, synthetic construct
  REGION 1..14: note = Description of sequence: r-tr[A5VI67]
  SITE 2: note = X refers to a non-epitope amino acid residue
  SITE 6: note = X refers to a non-epitope amino acid residue
  SITE 7: note = X refers to a non-epitope amino acid residue
  SITE 11: note = X refers to a non-epitope amino acid residue
  SITE 13: note = X refers to a non-epitope amino acid residue
  SITE 14: note = X refers to a non-epitope amino acid residue
KXKANXXFYKXDXX

SEQ ID NO: 44 - protein, 14 aa, synthetic construct
  REGION 1..14: note = Description of sequence: r-tr[A5VI67]
  SITE 2: note = X refers to a non-epitope amino acid residue
  SITE 6: note = X refers to a non-epitope amino acid residue
  SITE 7: note = X refers to a non-epitope amino acid residue
  SITE 11: note = X refers to a non-epitope amino acid residue
  SITE 13: note = X refers to a non-epitope amino acid residue
  SITE 14: note = X refers to a non-epitope amino acid residue
KXKVQXXFYKXDXX

SEQ ID NO: 45 - protein, 14 aa, synthetic construct
  REGION 1..14: note = Description of sequence: r-tr[A5VI67]
  SITE 2: note = X refers to a non-epitope amino acid residue
  SITE 6: note = X refers to a non-epitope amino acid residue
  SITE 7: note = X refers to a non-epitope amino acid residue
  SITE 11: note = X refers to a non-epitope amino acid residue
  SITE 13: note = X refers to a non-epitope amino acid residue
  SITE 14: note = X refers to a non-epitope amino acid residue
KXKMQXXFYKXDXX

SEQ ID NO: 46 - protein, 14 aa, synthetic construct
  REGION 1..14: note = Description of sequence: CH58 epitope region
KKKVHALFYKLDIV

SEQ ID NO: 47 - protein, 14 aa, synthetic construct
  REGION 1..14: note = Description of sequence: CH58 epitope region
  SITE 2: note = X refers to a non-epitope amino acid residue
  SITE 6: note = X refers to a non-epitope amino acid residue
  SITE 7: note = X refers to a non-epitope amino acid residue
  SITE 11: note = X refers to a non-epitope amino acid residue
  SITE 14: note = X refers to a non-epitope amino acid residue
KXKVHXXFYKXDIX

SEQ ID NO: 48 - protein, 694 aa, synthetic construct
  REGION 1..694: note = Description of sequence: BG505 T332N.DS.SOSIP.sc.664
MPMGSLQPLATLYLLGMLVASVLAAENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSACTQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPNNNTRKSIRIGPGQAFYATGDIIGDIRQAHCNVSKATWNETLGKVVKQLRKHFGNNTIIRFANSSGGDLEVTTHSFNCGGEFFYCNTSGLFNSTWISNTSVQGSNSTGSNDSITLPCRIKQIINMWQRIGQCMYAPPIQGVIRCVSNITGLILTRDGGSTNSTTETFRPGGGDMRDNWRSELYKYKVVKIEPLGVAPTRCKRRVVGGGSGGGGSGGGGSGGAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGGSGSGGSGSGLNDIFEAQKIEWHE

SEQ ID NO: 49 - protein, 679 aa, synthetic construct
  REGION 1..679: note = Description of sequence: 286.36.DS.SOSIP.sc.664
MPMGSLQPLATLYLLGMLVASVLAGEDLWVTVYYGVPVWKEANPTLFCASDAKAYKTEMHNVWATHACVPTDPNPQEMVLENVTEDFNMWKNGMVEQMHQDIISLWDQSLKPCVKLTPLCVTLNCTEVTRSSNGTINNNSTEMKNCSFNVTTDLRDKKKKEHALFYRLDIVPLDETNGTSSEYRLINCNTSTCTQACPKVSFDPIPIHYCAPAGYAILKCKDKKFNGTGPCKNVSTVQCTHGIKPVVSTQLLLNGSIAEGEIIIRSENLTNNAKIIIVQLNVTVEINCTRPNNNTRRSIRIGPGQTFYATGEIIGDIRQAHCNISREKWNRTLQKVEKKLEELFPNKTIHFTSSSGGDLEITTHSFNCMGEFFYCNTSALFNNNNDSTNSNITLPCRIRQFINMWQEVGRCMYAPPIQGVITCKSNVTGLLLTRDGGIINDTEIFRPGGGDMRDNWRSELYKYKVVEIKPLGIAPTTCKRRVVEGGSGGGGSGGGGSGGAVGIGAVFLGFLGAAGSTMGAASITLTAQARQLLSGIVQQQSNLLRAPEAQQHMLQLTVWGIKQLQTRVLAIERYLKDQQLLGIWGCSGKLICCTAVPWNGSWSNKSQDEIWHNMTWMQWDKEINNYTNIIYGLLEVSQNQQEKNEQDLLALDGSGGSGSGGSGSGLNDIFEAQKIEWHE

SEQ ID NO: 50 - protein, 703 aa, synthetic construct
  REGION 1..703: note = Description of sequence: 5768.04.DS.SOSIP.sc.664
MPMGSLQPLATLYLLGMLVASVLAADKLWVTVYYGVPVWKETTTTLFCASDARAYDTEVHNVWATHACVPTDPNPQEVVLGNVTENFNMWKNNMVEQMHEDIISLWDQSLKPCVRLTPLCVTLNCIDYYGNTTNSNNSSETMMEKGEIKNCSFNITTRLKDKMQKEYALFYKYDIVPIDNRVGNDTSNATSYRLTSCNTSVCTQACPKVSFEPIPIHYCAPAGFAILKCNDKKFNGTGPCKNVSTVQCTHGIKPVVSTQLLLNGSLAEEEVMIRSENFTDNAKTIIVQLNETVEINCTRPNNNTRKSIHMGPGKVFYTTGEIIGDIRQAHCNINRAKWNNTLIKIVEKLRVKFNKTISFKQSSGGDPEIEMHSFNCGGEFFYCNTTQLFNSTWFNNATLNVNSNVTEGSENITLPCRIRQIVNMWQEVGKCMYAPPIQGQIRCSSNITGLLLTRDGGGSNSSNTSEEVFRPGGGNMRDNWRSELYKYKVVKIEPLGIAPTKCKRRVVQGGSGGGGSGGGGSGGTVGIGALFLGFLGAAGSTMGAASMTLTVQARQLLSGIVQQQNNLLRAPQAQQHLLQLTVWGIKQLQARVLAVERYLKDQQLLGIWGCSGKLICCTAVPWNASWSNKSLNEIWDNMTWMEWEKEIDNYTSLIYTLIEESQNQQEKNEQELLELDGSGGSGSGGSGSGLNDIFEAQKIEWHE

SEQ ID NO: 51 - protein, 681 aa, synthetic construct
  REGION 1..681: note = Description of sequence: CZA97.DS.SOSIP.sc.664
MPMGSLQPLATLYLLGMLVASVLAVGNMWVTVYYGVPVWTDAKTTLFCASDTKAYDREVHNVWATHACVPTDPNPQEIVLENVTENFNMWKNDMVDQMHEDIISLWDQSLKPCVKLTPLCVTLHCTNATFKNNVTNDMNKEIRNCSFNTTTEIRDKKQQGYALFYRPDIVLLKENRNNSNNSEYILINCNASTCTQACPKVNFDPIPIHYCAPAGYAILKCNNKTFSGKGPCNNVSTVQCTHGIKPVVSTQLLLNGSLAEKEIIIRSENLTDNVKTIIVHLNKSVEIVCTRPNNNTRKSMRIGPGQTFYATGDIIGDIRQAYCNISGSKWNETLKRVKEKLQENYNNNKTIKFAPSSGGDLEITTHSFNCRGEFFYCNTTRLFNNNATEDETITLPCRIKQIINMWQGVGRCMYAPPIAGNITCKSNITGLLLVRDGGEDNKTEEIFRPGGGNMKDNWRSELYKYKVIELKPLGIAPTGCKRRVVEGGSGGGGSGGGGSGGAVGIGAVFLGFLGAAGSTMGAASLTLTVQARQLLSSIVQQQSNLLRAPEAQQHMLQLTVWGIKQLQTRVLAIERYLKDQQLLGIWGCSGKLICCTNVPWNSSWSNKSQTDIWNNMTWMEWDREISNYTDTIYRLLEDSQTQQEKNEKDLLALDGSGGSGSGGSGSGLNDIFEAQKIEWHE

SEQ ID NO: 52 - protein, 686 aa, synthetic construct
  REGION 1..686: note = Description of sequence: DU172.17.DS.SOSIP.sc.664
MKAKLLVLLCTFTATYAGNLWVTVYYGVPVWKEAKTTLFCASDAKAHKEEVHNIWATHACVPTDPNPQEIVLKNVTENFNMWKNDMVDQMHEDIISLWDQSLKPCVKLTPLCVTLNCSDVKIKGTNATYNNATYNNNNTISDMKNCSFNTTTEITDKKKKEYALFYKLDVVALDGKETNSTNSSEYRLINCNTSACTQACPKVSFDPIPIHYCAPAGYAILKCNNKTFNGTGPCNNVSTVQCTHGIKPVVSTQLLLNGSLAEEEVVIRFENLTNNAKIIIVHLNESVEINCTRPSNNTRKSVRIGPGQTFFATGDIIGDIRQAHCNISRKKWNTTLQRVKEKLKEKFPNKTIQFAPSSGGDLEITTHSFNCRGEFFYCYTSDLFNSTYMSNNTGGANITLQCRIKQIIRMWQGVGQCMYAPPIAGNITCKSNITGLLLTRDGGKEKNDTETFRPGGGDMRDNWRSELYKYKVVEIKPLGIAPDKCKRRVVEGGSGGGGSGGGGSGGAVGIGAVFLGFLGAAGSTMGAASMTLTVQARQLLSGIVQQQSNLLRAPEAQQHMLQLTVWGIKQLQTRVLAIERYLKDQQLLGIWGCSGKLICCTAVPWNASWSNKSYEEIWGNMTWMQWDREINNYTNTIYSLLEESQNQQEKNEKDLLALDGSGGSGSGGSGSGLNDIFEAQKIEWHE

SEQ ID NO: 53 - protein, 716 aa, synthetic construct
  REGION 1..716: note = Description of sequence: HT593.1.DS.SOSIP.sc.664
MPMGSLQPLATLYLLGMLVASVLATEKLWVTVYYGVPVWKEATTTLFCASDAKAYETEVHNVWATHACVPTDPNPQEVLLENVTENFNMWKNNMVEQMQEDIISLWDQSLKPCVKLTPLCVTLECHDVNVNGTANNGTTNVTESGVNSSDVTSNNVTNSNWGTMEKGEIKNCSFNITTNIRDKMQKETAQFYKLDIVPIEDQNKTNNTLYRLINCNTSVCTQACPKVSFEPIPIHYCTPAGFAILKCNDRNFNGTGPCKNVSTVQCTHGIKPVVSTQLLLNGSLAEAEVVIRSENFTNNAKTIIIQLNETVEINCTRPNNNTSKRISIGPGRAFRATKIIGNIRQAHCNISRATWNSTLKKIVAKLREQFGNKTIVFQPSSGGDPEIVMHSFNCGGEFFYCNTTQLFNSTWNSTEESNSTEEGTITLPCRIKQIINMWQEVGKCMYAPPIEGQIRCSSNITGLLLTRDGGNNNKTNGTEIFRPGGGDMRDNWRSELYKYKVVKIEPLGVAPTKCKRRVVQGGSGGGGSGGGGSGGAVGIVGAMFLGFLGAAGSTMGAASMTLTVQARLLLSGIVQQQNNLLRAPEAQQHLLQLTVWGIKQLQARVLAVERYLKDQQLLGIWGCSGKLICCTTVPWNTSWSNKSLSEIWDNMTWMQWEREIDNYTSLIYTLIEESQNQQEKNEQELLELDGSGGSGSGGSGSGLNDIFEAQKIEWHE

SEQ ID NO: 54 - protein, 694 aa, synthetic construct
  REGION 1..694: note = Description of sequence: KNH1209.18.DS.SOSIP.sc.664
MPMGSLQPLATLYLLGMLVASVLATDNLWVTVYYGVPVWKDAETTLFCASDAKAYATEKHNVWATHACVPTDPNPQEIHLENVTEEFNMWKNNMVEQMHTDIISLWDQSLKPCVKLTPLCVTLSCSNAKVSYSNATVNNTIQDEIKNCSFNTTTVLRDKRQKVYSLFYRLDIVQIDNSSSDSSSSEYRLINCNTSACTQACPKVTFEPIPIHYCAPAGFAILKCKDEEFNGTGPCKNVSTVQCTHGIKPVVSTQLLLNGSLAKREVKIRSENITNNAKNIIVQFVDPVEINCTRPNNNTRKSIHIGPGQAFYATGDIIGDIRQAHCNVSRSSWNKTLQQVAKQLGTYFKNKTIVFNTSSGGDPEITTHSFNCAGEFFYCDTSGLFNSSWNDTTWKESNSTGSNDTITLLCRIKQIINMWQRTGQCMYAPPIPGLISCKSNITGIILTRDGGNSHRTEETFRPGGGDMRDNWRSELYRYKVVQIEPLGVAPTRCRRRVVQGGSGGGGSGGGGSGGAVGIGAVFLGFLGAAGSTMGAASITLTVQARQLLSGIVQQQSNLLRAPEAQQHLLKLTVWGIKQLQARVLAVERYLRDQQLLGIWGCSGKLICCTNVPWNSSWSNKSYNDIWDNMTWLQWDKEIHNYTQLIYNLIEESQNQQEKNEQDLLALDGSGGSGSGGSGSGLNDIFEAQKIEWHE

SEQ ID NO: 55 - protein, 700 aa, synthetic construct
  REGION 1..700: note = Description of sequence: MB539.2B7.DS.SOSIP.sc.664
MPMGSLQPLATLYLLGMLVASVLAAENLWVTVYYGVPVWRDADTTLFCASDAKAYETEKHNVWATHACVPTDPNPQEIDLKNVTEEFNMWKNNMVEQMHTDIISLWDQSLKPCVKLTPLCVTLNCSNANVTSENSTIMGDREEIKNCSFNMTTELRDKRQKVYSLFYRLDVVQINENQGNSSNNNYSEYRLINCNTSACTQACPKVSFEPIPIHYCAPAGFAILKCKDEEFNGTGPCKNVSTVQCTHGIKPVVSTQLLLNGSTAEKEIKIRSENITNNAKIIIVQLVKPVIINCTRPNNNTRRSVHIGPGQAFYATGDIIGNIRQAYCTVNRTDWNNTLQQVAKQLGKHFENKTIIFTKSSGGDLEITTHSFNCGGEFFYCNTSSLFNSTWSHNNSTLLGSNSTESNETITLPCRIKQIVNMWQRTGQCMYAPPIKGVIMCVSNITGLILTRDGGNDNSTNENETFRPGGGDMRDNWRSELYKYKVVQIEPLGVAPTRCKRRVVEGGSGGGGSGGGGSGGAVGIGAVFLGFLGAAGSTMGAASITLTVQARQLLSGIVRQQSNLLRAPEAQQHLLKLTVWGIKQLQARVLAVERYLRDQQLLGIWGCSGKLICCTSVPWNSSWSNKSLDEIWENMTWLQWEKEINNYTGLIYSLLEESQNQQEKNEQDLLALDGSGGSGSGGSGSGLNDIFEAQKIEWHE

SEQ ID NO: 56 - protein, 694 aa, synthetic construct
  REGION 1..694: note = Description of sequence: BG505 K169E SOSIP.664.sc
MPMGSLQPLATLYLLGMLVASVLAAENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKEQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPNNNTRKSIRIGPGQAFYATGDIIGDIRQAHCNVSKATWNETLGKVVKQLRKHFGNNTIIRFANSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGGGSGGGGSGGGGSGGAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGGSGSGGSGSGLNDIFEAQKIEWHE

SEQ ID NO: 57 - protein, 694 aa, synthetic construct
  REGION 1..694: note = Description of sequence: BG505 D368R+N279K SOSIP.664.sc (DKO)
MPMGSLQPLATLYLLGMLVASVLAAENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVMIRSENITKNAKNILVQFNTPVQINCTRPNNNTRKSIRIGPGQAFYATGDIIGDIRQAHCNVSKATWNETLGKVVKQLRKHFGNNTIIRFANSSGGRLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGGGSGGGGSGGGGSGGAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGGSGSGGSGSGLNDIFEAQKIEWHE

SEQ ID NO: 58 - protein, 694 aa, synthetic construct
  REGION 1..694: note = Description of sequence: BG505 N332T SOSIP.664.sc
MPMGSLQPLATLYLLGMLVASVLAAENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPNNNTRKSIRIGPGQAFYATGDIIGDIRQAHCTVSKATWNETLGKVVKQLRKHFGNNTIIRFANSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGGGSGGGGSGGGGSGGAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGGSGSGGSGSGLNDIFEAQKIEWHE

SEQ ID NO: 59 - protein, 396 aa, synthetic construct
  REGION 1..396: note = Description of sequence: EF-Tu 396
MAEKEHYERTKPHVNIGTIGHVDHGKTTLTAAITKVLAAKGLAKAEDYADIDAAPEEKERGITINTAHVEYETEKRHYAHIDAPGHADYVKNMITGAAQMDGAILVVAATDGPMPQTREHILLARQVGVQYIVVFLNKTDLVDDDELVDLVEMEVRDLLSEYDFPGDDVPVVRGSALKALEGDPEQEKVILHLMDVIDDYIPTPKRPTDKPFMMPVEDVFTITGRGTVASGRIDRGTVKVGDEVEIVGLTEDVLKSTVTGLEMFHKTLDLGEAGDNVGVLLRGISHDQIQRGQVLAEPGSIQTHKNFKGEVYVMTKEEGGRHTPFFSNYRPQFYFHTTDVTGTIELPDGVEMVMPGDNVTFTVNLQKPVALEKGLKFTIREGGHTVGAGVVSDILD

SEQ ID NO: 60 - DNA, 1191 bp, synthetic construct
  misc_feature 1..1191: note = Description of sequence: EF-Tu 1191bp
  source 1..1191: mol_type = other DNA
atggctgaaaaagaacattatgaacgtacaaaaccccacgttaacattggtactattggccacgttgaccacgggaagactactttaactgctgctattacaaaggtattggcagccaaaggtttagcaaaggcagaagattacgctgatatcgatgctgctccagaagaaaaggaacgtggtatcactatcaacactgcccacgttgaatacgaaactgaaaagcgtcactacgcacacatcgatgcccctggacacgctgactacgttaagaacatgatcactggtgctgcacaaatggatggtgctatccttgttgttgctgctactgatggtcctatgccacaaactcgtgaacacattcttcttgctcgtcaggttggtgttcaatacatcgttgtattcttgaacaagactgacttagttgacgatgatgaattagttgacttagttgaaatggaagttcgtgacttacttagcgaatacgatttccctggtgacgatgttccagttgttcgtggatctgctcttaaggcgcttgaaggtgacccagaacaagaaaaagttatccttcacttaatggatgttattgatgactacatcccaactccaaagcgtcctactgacaagccattcatgatgcctgtcgaagacgtatttactatcactggtcgtggtactgttgcttctggtcgtattgaccgtggtactgttaaggtcggtgacgaagttgaaatcgttggtttaactgaagacgttcttaagtcaactgttactggtttggaaatgttccacaagactcttgatcttggtgaagccggtgataacgttggtgtacttcttcgtggtatttcacacgaccaaatccaacgtggtcaagttttggctgaaccaggttcaatccaaactcacaagaacttcaagggtgaagtttacgttatgactaaggaagaagggggacgtcacactccattcttctcaaactaccgtccacaattctacttccacacaactgatgttactggtactatcgaattaccagatggtgtagaaatggttatgcctggtgataacgttacattcactgttaacttacaaaagccagttgctcttgaaaagggtcttaagttcactattcgtgaaggtggacacactgttggtgccggtgttgtatccgccatcttagactaa